(19) United States
(12) Patent Application Publication
(10) Pub. No.: US 2003/0003112 A1
(43) Pub. Date: Jan. 2, 2003

(54) Porcine reproductive and respiratory syndrome virus (PRRSV) recombinant poxvirus vaccine

(76) Inventors: Jean-Christophe Audonnet, Lyon (FR); Michel Bublot, Delmar, NY (US); Jennifer Perez, Worcester, MA (US); Philippe Baudu, Craponne (FR)

Correspondence Address: Thomas J. Kowalski, FROMMER LAWRENCE & HAUG LLP, 745 Fifth Avenue, New York, NY, 10151, US

(21) Appl. No.: 09/862,393

(22) Filed: May 21, 2001

Related U.S. Application Data
(60) Provisional application No. 60/206,655, filed on May 24, 2000.

(51) Int. Cl. A61K 39/285; A61K 39/275; C12N 7/00

(52) U.S. Cl. 424/232.1; 424/186.1; 435/235.1

(57) ABSTRACT

What is described is a recombinant vector, such as a virus; for instance, a poxvirus, such as avipox virus, containing foreign DNA from porcine reproductive and respiratory syndrome virus. What are also described are immunological compositions containing the recombinant poxvirus for inducing an immunological response in a host animal to which the immunological composition is administered. Also described are methods of treating or preventing disease caused by porcine reproductive and respiratory syndrome virus by administering the immunological compositions of the invention to an animal in need of treatment or susceptible to infection by porcine reproductive and respiratory syndrome virus.

### CROSS REFERENCE TO RELATED APPLICATIONS

This application claims priority from U.S. application Ser. No. 60/206,655, filed May 24, 2000. Reference is made to WO 98/03658, published Jan. 29, 1998 from PCT/FR97/01313, filed Jul. 15, 1997 and designating the U.S. and claiming priority from French application 96 09338, filed Jul. 19, 1996; the U.S. continuation-in-part of PCT/FR97/01313, namely, U.S. application Ser. No. 09/232,468, filed Jan. 15, 1999 for “POLYNUCLEOTIDE VACCINE FORMULA AGAINST PORCINE REPRODUCTIVE AND RESPIRATORY PATHOLOGIES”; now U.S. Pat. No. 6,207,165, and, U.S. application Ser. No. 60/151,564, filed Aug. 31, 1999. Each of these applications and each document cited or referenced in each of these applications (“application cited documents”), and each document cited or referenced in application cited documents, are hereby incorporated herein by reference. The present invention and/or nucleic acid molecules disclosed herein can be employed with or in conjunction with inventions or teachings of the foregoing applications.

### FIELD OF THE INVENTION

The present invention relates to recombinant vectors, such as viruses, such as poxviruses, and to methods of making and using the same. The invention further relates to recombinant avipoxes such as ALVAC, which virus expresses gene products of Porcine Reproductive and Respiratory Syndrome Virus (PRRSV); for instance, a recombinant vector such as a virus, e.g., an ALVAC recombinant, that contains and expresses PRRSV ORFs 3 and 5, or 5 and 6, or 4 and 5, or their combinations in particular 3 and 5 and 6, or 4 and 5 and 6. The invention also relates to immunological compositions or vaccines which induce an immune response directed to PRRSV gene products. The invention yet further relates to such compositions or vaccines and which confer protective immunity against infection by PRRSV. And, the invention relates to methods for making and using such recombinant vectors and compositions.

Several publications are referenced in this application. Full citation to these documents is found at the end of the specification preceding the claims, and/or where the document is cited. These documents pertain to the field of this invention; and, each of the documents cited or referenced in this application (“herein cited documents”), and each document cited or referenced in herein cited documents, are hereby incorporated herein by reference.

### BACKGROUND OF THE INVENTION

Porcine reproductive and respiratory syndrome virus (PRRSV) belongs to a family of enveloped positive-strand RNA viruses called arteriviruses. Other viruses in this family are the prototype virus, equine arteritis virus (EAV), lactate dehydrogenase-elevating virus (LDV) and simian hemorrhagic fever virus (SHFV) (de Vries et al., 1997 for review). Striking features common to the Coronaviridae and Arteriviridae have recently resulted in their placement in a newly created order, Nidovirales (Pringle, 1996; Cavanagh, 1997; de Vries et al., 1997). The four members of the Arterivirus group, while being similar in genome organization, replication strategy and amino acid sequence of the proteins are also similar in their preference for infection of macrophages, both in vivo and in vitro (Conzelmann et al., 1993; Meulenberg et al., 1993a).

A new viral disease of pigs was detected in North America in 1987 (Hill, 1990) and in Europe in 1990 (Paton et al. 1991). The disease, variously known as porcine reproductive and respiratory syndrome (PRRS), swine infertility and respiratory syndrome (SIRS), porcine epidemic abortion and respiratory syndrome (PEARS) and mystery swine disease is mainly characterized by reproductive failure in sows and respiratory problems in pigs of all ages. The causative agent, now known as PRRS virus, was first isolated in The Netherlands as Lelystad virus (Wensvoort et al., 1991). Subsequently, Benfield et al. (1992) and Collins et al. (1992) isolated a related virus in North America (prototype strain ATCC VR2332). Polyvalent antisera specific for European isolates of PRRSV cross-react with North American isolates in an immunoperoxidase assay on infected macrophages and vice versa (Wensvoort et al., 1992); however, further studies indicate that European and North American isolates represent two distinct genotypes that have evolved independently on separate continents (Mardassi et al., 1995: Meng et al., 1995a and b; Murtaugh et al., 1995; Nelsen et al., 1999). The near-simultaneous global emergence of a new swine disease caused by divergently evolved viruses suggests that changes in swine husbandry and management may have contributed to the emergence of PRRS (Nelsen et al., 1999).

In the USA, 40 to 60% of herds are currently estimated to be infected (Bautista et al., 1993; Cho et al., 1993) while in Europe, PRRSV infection is believed to have affected greater than 50% of herds in some areas (Albina, 1997). It is difficult to gauge the economic effect of the disease, which varies from country to country. In the USA, losses of $500 per sow per year have been documented (Done and Paton, 1995).

Two main groups of clinical signs are associated with the occurrence of PRRS although it is now recognized that clinical effects vary greatly among infected herds and in many cases, infection is sub-clinical and productivity is within acceptable parameters. The two groups are: (1) Reproductive signs which include premature births, late-term abortions, piglets born weak and increased numbers of still-births and mummifications (Done and Paton, 1995); (2) Signs of respiratory disease in neonatal pigs which include labored breathing and coughing as the most dominant characteristics. The symptoms usually occur in pigs about three weeks of age though all ages are susceptible. In contrast to the reproductive failures, clinically overt respiratory disease is harder to reproduce experimentally (Zimmermann et al. 1997).

These clinical signs vary considerably and may be influenced by the virus strain (Halbur et al., 1995), age at infection and differences in genetic susceptibility (Halbur et al., 1992), concurrent infections (Galina et al., 1994), pig density, pig movements and housing systems (Done et al., 1996) and immune status including the presence of low levels of PRRS virus-specific antibodies which may be enhancing (Yoon et al., 1994).

In terms of pathogenesis, the most significant change induced by PRRSV is the severe damage to alveolar macrophages, which are destroyed in huge numbers (reviewed in Done and Paton, 1995; Rossow, 1998). The induction of apoptosis in a large number of mononuclear cells in the lungs and lymph nodes might be an explanation for a dramatic reduction in the number of alveolar macrophages and circulating lymphocytes and monocytes in PRRSV-infected pigs (Sirinarumitr et al., 1998; Sur et al., 1998). Coupled with the destruction of circulating lymphocytes and the destruction of the mucociliary clearance system, this may suppress immunity and render pigs more susceptible to secondary infection. An enhanced rate of bacterial secondary infections has been documented following PRRSV infection (Galina et al., 1994; Done and Paton, 1995; Nakamine et al. 1998). The severity of PRRSV infection may be also increased by bacterial or mycoplasma infection (Thacker et al. 1999). In addition a number of viral infections have been found associated with PRRS (Carlson, 1992; Brun et al., 1992; Halbur et al., 1993; Done et al., 1996; Heinen et al., 1998).

There appear to be three routes of transmission: (1) nose to nose or close contact (Done et al., 1996), (2) aerosols (Le Potier et al., 1995), and (3) spread through urine, feces and semen. Transmission via insemination with contaminated semen is now well documented (Yeager et al., 1993; Albina, 1997).

The genome organization of arteriviruses is reviewed in de Vries et al. (1997). The genome RNA is single-stranded, infectious, polyadenylated and 5′ capped. The genome of PRRSV is small, at 15,088 bases. Both the EAV and LDV genomes are slightly smaller at 12,700 bases and 14,200 bases, respectively. Complete sequences of EAV, LDV and PRRSV genomes are available (Den Boon et al., 1991; Godeny et al., 1993; Meulenberg et al. 1993a).

The genome contains eight open reading frames (ORFs) that encode, in the following order, the replicase genes (ORFs 1a and 1b), the envelope proteins (ORFs 2 to 6) and the nucleocapsid protein (ORF 7) (Meulenberg et al. 1993a). ORFs 2 to 7 are expressed from six sub-genomic RNAs, which are synthesized during replication (Meng et al., 1994, 1996). These sub-genomic RNAs form a 3′ co-terminal nested set and are composed of a common leader, derived from the 5′ end of the viral genome (Meulenberg et al. 1993b). Although the RNAs are structurally polycistronic, translation is restricted to the unique 5′ sequences not present in the next smaller RNA of the set. Two large overlapping open reading frames (ORFs), designated ORF 1a and ORF 1b, take up more than two thirds of the genome. The second ORF, ORF 1b is only expressed after a translational read-through via a −1 frame shift mediated by a pseudoknot structure (Brierley 1995). The polypeptides encoded by these ORFs are proteolytically cleaved by virus-encoded proteases to yield the proteins involved in RNA synthesis.

The current knowledge of the characteristics of the protein encoded by each ORF is summarized below. The amino acid homologies for ORFs 2 through 7 of the American PRRSV isolate VR-2332 as compared to the Lelystad virus, LDV and EAV have been described (Murtaugh, 1995).

ORF 2 encodes a 29-30 kDa N-glycosylated structural protein (GP2 or GS) showing the features of a class 1 integral membrane glycoproteins (Meulenberg and Petersen-den Besten, 1996 using the Ter Huurne strain of Lelystad virus). The ORF 2 protein shows 63% amino acid homology when the American VR-2332 isolate is compared to Lelystad virus (Murtaugh et al., 1995). ORF 3 encodes an N-glycosylated 45-50 kDa minor structural protein designated GP3 (van Nieuwstadt et al., 1996). This is the least conserved ORF when comparing VR-2332 to Lelystad virus with only 58% amino acid identity between the two viruses (Murtaugh et al. 1995). Recent data indicated that GP3 is a non-structural glycoprotein that is released from the infected cells in a soluble form (Gonin et al., 1998).

ORF 4 encodes a 31-35kDA minor N-glycosylated membrane protein designated GP4 (van Nieuwstadt et al., 1996). Monoclonal antibodies against GP4 are neutralizing indicating that at least part of the protein should be on the virion surface—the monoclonals are at least partially cross reactive with European isolates (van Nieuwstadt et al., 1996). The VR-2332 ORF 4 protein shows 68% amino acid identity when compared with Lelystad virus, and contains five putative membrane spanning domains (Murtaugh et al., 1995).

ORF 5 encodes GP5 or GL, which is a 25 kDa major envelope glycoprotein (Meulenberg et al., 1995). The PRRS GP5 protein has been demonstrated to be an efficient inducer of apoptosis although the mechanism has not been determined (Suarez et al. 1996). When comparing the ORF 5 protein of PRRS Lelystad virus and the American VR-2332 isolate, 59% amino acid homology is found (Murtaugh et al. 1995). One region between residues 26 and 39 was found to correspond to a hypervariable region which involved 0 to 3 potential N-glycosylation sites (Pirzadeh et al., 1998b). The protein appears to be poorly immunogenic since it has been difficult to raise monoclonal antibodies against it by standard means (Pirzadeh and Dea, 1997; Drew et al., 1995). The protein is, however, recognized by most convalescent pig sera (Nelson et al., 1993; Meulenberg et al., 1995). Seroneutralization of PRRSV correlates with antibody response to the GP5 major envelope glycoprotein (Gonin et al., 1999). Monoclonal antibodies against GP5 have neutralizing activity which is usually specific for the parent virus only (Pirzadeh and Dea, 1997; Weiland et al., 1999).

ORF 6 encodes an 18 kDA class III non-glycosylated integral membrane (M) protein (Meulenberg et al., 1995). Topographical studies with PRRSV, LDV and EAV have indicated that the ORF 5 (GL) protein and the ORF 6 M protein are present in the virion as heterodimers covalently linked by disulfide bonds (Mardassi et al. 1996; de Vries et al., 1995b; Faaberg et al., 1995). The bonds probably exist between cysteine residues in the ectodomains of the two proteins that are highly conserved in the equivalent proteins of PRRSV and EAV (Plagemann, 1996, Meulenberg et al., 1993a). In LDV it has been demonstrated that cleavage of the disulfide bond in virions results in loss of infectivity, perhaps indicating that the linkage of these two proteins may stabilize the virus attachment site for interaction with host cell receptor (Faaberg and Plagemann, 1995). The ORF 6 protein is the most conserved protein when comparing amino acid homologies between Lelystad virus and isolate VR-2332 with 78% homology (Murtaugh et al., 1995). Recent data suggest that the product of ORF 6 has a major role in cellular immunity (Bautista et al., 1999).

ORF 7 encodes a 15 kDa non-glycosylated basic protein—thought to be the nucleocapsid protein (N) based on similarity of sequence to other nucleocapsid proteins. The ORF 7 protein of Lelystad virus has 65% amino acid identity between Lelystad virus and American isolate VR-2332 (Murtaugh et al., 1995). The N protein appears to be the most immunogenic PRRSV protein as antibody to N is the first to appear as detected by Western blot and is the most persistent (Nelson et al., 1994, Yoon et al., 1995).

A new open reading frame conserved in arteriviruses has been recently identified in the equine arteritis virus (EAV) genome (Snijder et al., 1999). This EAV ORF was designated 2a and is coding for an essential 8kDa structural protein called “E”. In PRRSV, the homologous ORF has been designated 2b, the ORF 2 coding for GP2 (see above) being renamed ORF 2a (Snijder et al., 1999).

It is not yet clear what constitutes a protective immune response to PRRSV. In infected pigs, viremia can persist for many weeks in the face of circulating antibody and little is known about the mechanisms by which immunity develops. However, in herds where PRRSV persists, sows do not suffer repeated reproductive losses, indicating some form of protective immunity does develop (Done et al., 1996; Rossow et al., 1998). Neutralizing antibodies can decline quickly, as fast as 6 months after initial infection (Ohlinger et al. 1992) possibly indicating a short duration of active immunity. PRRSV can replicate in and spread from pigs with neutralizing antibodies, indicating that serum-neutralizing antibodies are not necessarily an essential part of the immune response (Ohlinger 1995). Exposure of swine to enzootic PRRSV will provide protection against homologous PRRSV-induced reproductive losses, but the extent and duration of protection against heterologous PRRSV may be variable and dependent on antigenic relatedness of the virus strains used for inoculation and challenge-exposure (Lager et al., 1999). Anti-PRRSV cellular immunity has been detected in infected pigs (Rossow. 1998). The proliferation T cell responses to the structural polypeptides of PRRSV have been recently analyzed using vaccinia recombinant viruses expressing structural polypeptides (ORFs 2 to 7; see below). The greater response was observed to the product of ORF 6 (Bautista et al., 1999).

Both killed and live attenuated virus vaccines are available. Plana Duran (1997) has described a killed oil-adjuvanted vaccine that induced 70% protection. In the USA, Gorcyca et al (1995) has described a live attenuated vaccine, which is administered as a single intramuscular injection. Although producing a detectable viremia, it was shown to be safe in late gestation sows, and was not transmitted to susceptible contact pigs. Field trials indicated there was significant benefit in use of the vaccine in nursery pigs in units with endemic PRRSV infection (Done et al., 1996). However, other studies indicated a lack of safety or efficacy of live attenuated vaccine if administered during gestation (Dewey et al., 1999; Mengeling et al., 1999b). Furthermore, in some swine herds, the vaccine strain may have persisted and mutated to a less attenuated form (Mengeling et al., 1999a). A recent study showed that some strains of PRRSV now circulating in US swine herds are more virulent than those encountered in the past. Clinical PRRS in vaccinated herds suggests need for a new generation of vaccines (Mengenling et al., 1998). Another recent study showed that European serotype PRRSV vaccine protects against European serotype challenge whereas an American serotype vaccine does not (van Woensel et al. 1998a and b). In summary, there is a need for new improved vaccines at both safety and efficacy levels.

Wensvoort, U.S. Pat. No. 5,620,691 or WO 92/21375 relates to “Leylstad Agent” as the “causative agent of “Mystery Swine Disease”; and provides a deposit of this agent (I-1102, deposited Jun. 5, 1991 with the Institut Pasteur, Paris, France), and certains open reading frames of it. While Wensvoort may mention that the agent or parts of it could be incorporated in a vector system such as vaccinia virus, herpesvirus, pseudorabies virus, or adenovirus, there is no teaching or suggestion in Wensvoort of how to construct a recombinant vector system containing and expressing a PRRSV immunogen or epitope of interest, as in the present invention, and there is especially no teaching or suggestion of a recombinant avipox virus containing and expressing a PRRSV ORF, or the particular combinations of ORFs and/or recombinants of the present invention.

Plana Duran et al. (1997) relates to the expression of ORFs 2 through 7 of a Spanish isolate of PRRSV in a baculovirus expression system. ORFs 3 through 7 were tested for immunogenicity in pregnant sows. Only vaccination with ORF 7 expressing the N protein gave a significant antibody response (immunoperoxidase monolayer assay). Immunization with ORF 7 gave no protection against challenge, however ORFs 3 and 5, alone or in combination, gave 50 to 70% protection against reproductive losses (Plana Duran et al. 1997). Piglets from mothers receiving ORFs 3 and 5 also demonstrated no anti-PRRSV antibody post-weaning after challenge indicating the absence of virus replication.

Pirzadeh and Dea (1998) vaccinated pigs with a plasmid DNA expressing ORF 5. Induction of neutralizing antibodies, lymphocyte proliferation and protection against PRRSV challenge were observed. Similarly, Kwang et al. (1999) evaluated the immunogenicity of plasmid DNA encoding ORFs 4 to 7 in pigs. DNA immunization against PRRS virus results in the production of both humoral and cell mediated immune responses in 71 % and 86% immunized pigs, respectively. The results also indicate that neutralization epitopes for PRRS virus are present on the viral envelope glycoproteins encoded by ORF 4 and ORF 5.

Cochran, U.S. Pat. No. 6,033,904 or WO 96/22363 relates to recombinant swinepox virus and mentions PRRSV ORF 7; but, Cohcran fails to teach or suggest a recombinant vector system containing and expressing a PRRSV immunogen or epitope of interest, as in the present invention, and there is especially no teaching or suggestion of a recombinant avipox virus containing and expressing a PRRSV ORF, or the particular combinations of ORFs and/or recombinants of the present invention.

Cochran, WO 00/03030 concerns recombinant raccoonpox virus and mentions PRRSV but, Cohcran fails to teach or suggest a recombinant vector system containing and expressing a PRRSV immunogen or epitope of interest, as in the present invention, and there is especially no teaching or suggestion of a recombinant avipox virus containing and expressing a PRRSV ORF, or the particular combinations of ORFs and/or recombinants of the present invention.

Vaccinia virus has been used successfully to immunize against smallpox, culminating in the worldwide eradication of smallpox in 1980. With the eradication of smallpox, a new role for poxviruses became important, that of a genetically engineered vector for the expression of foreign genes (Panicali and Paoletti, 1982; Paoletti et al., 1984). Genes encoding heterologous immunogens have been expressed in vaccinia, often resulting in protective immunity against challenge by the corresponding pathogen (reviewed in Tartaglia et al., 1990). A highly attenuated strain of vaccines, designated MVA, has also been used as a vector for poxvirus-based vaccines. Use of MVA is described in U.S. Pat. No. 5,185,146.

Two additional vaccine vector systems involve the use of naturally host-restricted poxviruses, avipox viruses. Both fowlpoxvirus (FPV; Taylor et al. 1988a, b) and canarypoxvirus (CPV; Taylor et al., 1991 & 1992) have been engineered to express foreign gene products. Fowlpox virus (FPV) is the prototypic virus of the Avipox genus of the Poxvirus family. The virus causes an economically important disease of poultry which has been well controlled since the 1920's by the use of live attenuated vaccines. Replication of the avipox viruses is limited to avian species (Matthews, 1982) and there are no reports in the literature of avipoxvirus causing a productive infection in any non-avian species including man. This host restriction provides an inherent safety barrier to transmission of the virus to other species and makes use of avipoxvirus based vaccine vectors in veterinary and human applications an attractive proposition.

FPV has been used advantageously as a vector expressing immunogens from poultry pathogens. The hemagglutinin protein of a virulent avian influenza virus was expressed in an FPV recombinant (Taylor et al., 1988c). After inoculation of the recombinant into chickens and turkeys, an immune response was induced which was protective against either a homologous or a heterologous virulent influenza virus challenge (Taylor et al., 1988c). FPV recombinants expressing the surface glycoproteins of Newcastle Disease Virus have also been developed (Taylor et al., 1990; Edbauer et al., 1990).

Other attenuated poxvirus vectors have been prepared by genetic modifications of wild type strains of virus. The NYVAC vector, derived by deletion of specific virulence and host-range genes from the Copenhagen strain of vaccinia (Tartaglia et al., 1992), has proven useful as a recombinant vector in eliciting a protective immune response against an expressed foreign immunogen.

Another engineered poxvirus vector is ALVAC, derived from canarypox virus. ALVAC does not productively replicate in non-avian hosts, a characteristic thought to improve its safety profile (Taylor et al., 1991 & 1992). Both ALVAC and NYVAC are BSL-1 vectors.

One approach to the development of a subunit PRRSV vaccine is the use of live viral vectors to express relevant PRRSV ORFs. Recombinant poxviruses can be constructed in two steps known in the art and analogous to the methods for creating synthetic recombinants of poxviruses such as the vaccinia virus and avipox virus described in U.S. Pat. Nos. 4,769,330; 4,722,848; 4,603,112; 5,110,587; 5,174,993; 5,494,807; 5,942,235, and 5,505,941, the disclosures of which are incorporated herein by reference. It can thus be appreciated that provision of a PRRSV recombinant poxvirus, and of compositions and products therefrom particularly ALVAC based PRRSV recombinants and compositions and products therefrom, especially such recombinants expressing ORFs 2, 3, 4, 5, 6, or 7 or any combination thereof of PRRSV, and compositions and products therefrom would be a highly desirable advance over the current state of technology.

### OBJECTS AND SUMMARY OF THE INVENTION

An object of this invention can be any one or all of: providing recombinant viruses, compositions and methods for treatment and prophylaxis of infection by PRRSV, as well as methods for making such viruses.

The invention provides a recombinant vector, such as a recombinant virus, e.g., a recombinant poxvirus, that contains and expresses at least one exogenous nucleic acid molecule; and, the at least one exogenous nucleic acid molecule can comprise a nucleic acid molecule encoding an immunogen or epitope of interest from PRRSV or can be an ORF or portion thereof of PRRSV. The invention further provides immunological (or immunogenic), or vaccine compositions comprising such a virus or the expression product(s) of such a virus. The invention further provides methods for inducing an immunological (or immunogenic) or protective response against PRRSV, as well as methods for preventing or treating PRRSV or disease state(s) caused by PRRSV, comprising administrering the virus or an expression product of the virus, or a composition comprising the virus, or a composition comprising an expression product of the virus. The invention also comprehends expression products from the virus as well as antibodies generated from the expression products or the expression thereof in vivo and uses for such products and antibodies, e.g., in diagnostic applications.

The term “comprising” in this disclosure can mean “including” or can have the meaning commonly given to the term “comprising” in U.S. Patent Law.

These and other embodiments are disclosed or are obvious from and encompassed by the following detailed description.

### BRIEF DESCRIPTION OF THE DRAWINGS

A better understanding of the present invention will be had by referring to the accompanying drawings, incorporated herein by reference, in which:

FIG. 1 (SEQ ID NO: 1) shows the nucleotide sequence of a 3.7 kilobase pair fragment of ALVAC DNA containing the C6 open reading frame.

FIG. 2 shows the map of pJP 115 donor plasmid.

FIG. 3 (SEQ ID NO: 10) shows the nucleotide sequence of the 2.6 kilobase pair fragment from pJP115 donor plasmid from the KpnI (position 653) to the SacI (position 3214) restriction sites.

FIG. 4 shows the map of pJP119 donor plasmid.

FIG. 5 (SEQ ID NO: 14) shows the nucleotide sequence of the 2.6 kilobase pair fragment from pJP119 donor plasmid from the KpnI (position 653) to the SacI (position 3262) restriction sites.

FIG. 6 shows the map of pJP103 donor plasmid.

FIG. 7 (SEQ ID NO: 19) shows the nucleotide sequence of the 2.4 kilobase pair fragment from pJP103 donor plasmid from the KpnI (position 653) to the SacI (position 3016) restriction sites.

FIG. 8 shows the map of pJP110 donor plasmid.

FIG. 9 (SEQ ID NO: 26) shows the nucleotide sequence of the 2.4 kilobase pair fragment from pJP110 donor plasmid from the KpnI (position 653) to the SacI (position 3064) restriction sites.

FIG. 10 shows the map of pJP 100 donor plasmid.

FIG. 11 (SEQ ID NO : 31) shows the nucleotide sequence of the 2.3 kilobase pair fragment from pJP100 donor plasmid from the KpnI (position 653) to the SacI (position 2986) restriction sites.

FIG. 12 shows the map of pJP113 donor plasmid.

FIG. 13 (SEQ ID NO: 32) shows the nucleotide sequence of the 3.1 kilobase pair fragment from pJP1 13 donor plasmid from the KpnI (position 653) to the SacI (position 3732) restriction sites.

FIG. 14 shows the map of pJP101 donor plasmid.

FIG. 15 (SEQ ID NO: 37) shows the nucleotide sequence of the 2.2 kilobase pair fragment from pJP101 donor plasmid from the KpnI (position 653) to the SacI (position 2851) restriction sites.

### DETAILED DESCRIPTION

In one aspect, the present invention provides an immunological or vaccine composition or a therapeutic composition for inducing an immunological or protective response in a host animal inoculated with the composition. The composition includes a carrier or diluent or excipient and/or adjuvant, and a recombinant vector, such as a recombinant virus. The recombinant virus can be a modified recombinant virus; for instance, a recombinant of a virus that has inactivated therein (e.g., disrupted or deleted) virus-encoded genetic functions. A modified recombinant virus can have inactivated therein virus-encoded nonessential genetic functions; for instance, so that the recombinant virus has attenuated virulence and enhanced safety. The virus used in the composition according to the present invention is advantageously a poxvirus, such as a vaccinia virus or preferably an avipox virus, e.g., a fowlpox virus or more preferably a canarypox virus; and more advantageously, an ALVAC virus. It is advantageous that the recombinant vector or recombinant virus have expression without replication in mammalian species. The recombinant vector or modified recombinant virus can include, e.g., within the virus genome, such as within a non-essential region of the virus genome, a heterologous DNA sequence that encodes an immunogenic protein, e.g., derived from PRRSV ORF(s), e.g., PRRSV ORF 2, 3, 4, 5, 6, or 7 or any combination thereof, preferably PRRSV ORFs 3 and 5, or 5 and 6, or 4 and 5, or their combinations in particular 3 and 5 and 6, or 4 and 5 and 6. (wherein the immunogenic protein can be an epitope of interest, e.g., an epitope of interest from a protein expressed by any one or more of PRRSV ORF 2, 3, 4, 5, 6 or 7, e.g., an epitope of interest from PRRSV ORFs 3 and 5, or 5 and 6, or 4 and 5, or their combinations in particular 3 and 5 and 6, or 4 and 5 and 6.).

It is advantageous that the recombinant vector or recombinant virus contains and expresses PRRSV ORFs 3 and 5, or 5 and 6, or 4 and 5, or their combinations in particular 3 and 5 and 6, or 4 and 5 and 6. In another aspect, the invention provides the in vitro expression of these ORFs by a recombinant vector or recombinant virus. The expression product can then be isolated and employed in diagnostic or therapeutic or immunological or immunogenic or vaccine compositions, e.g., admixed with a suitable carrier, excipient, diluent and/or adjuvant. In a further aspect, the invention advantageously provides the in vivo expression of these ORFs and compositions comprising a recombinant vector or recombinant virus that contains and expresses these ORFs. Such compositions can contain the recombinant vector or recombinant virus and a suitable carrier, excipient, diluent and/or adjuvant. The invention further provides methods for obtaining a therapeutic, immunogenic, immunological and/or protective response comprising administering such a recombinant vector or recombinant virus that contains and expresses these ORFs and/or a composition comprising such a recombinant vector or recombinant virus and/or the in vitro expression products of these ORFs and/or a composition comprising such expression products.

In yet another aspect, the present invention provides an immunogenic composition containing a recombinant vector such as a recombinant virus, e.g., a modified recombinant virus having inactivated (e.g., deleted or disrupted) virus-encoded genetic functions, such as nonessential virus-encoded genetic functions, so that the recombinant virus has attenuated virulence and enhanced safety. The modified recombinant virus includes, within the virus genome, e.g., within a non-essential region of the virus genome, a heterologous DNA sequence that encodes an immunogenic protein (e.g., derived from PRRSV ORFs, e.g., PRRSV ORF 2, 3, 4, 5, 6, or 7, preferably, PRRSV ORFs 3 and 5, or 5 and 6, or 4 and 5, or their combinations in particular 3 and 5 and 6, or 4 and 5 and 6.) wherein the composition, when administered to a host, is capable of inducing an immunological response specific to the immunogenic protein (for instance, wherein the immunogenic protein can be an epitope of interest, e.g., an epitope of interest from a protein expressed by any one or more of PRRSV ORF 2, 3, 4, 5, 6 or 7, preferably PRRSV ORFs 3 and 5, or 5 and 6, or 4 and 5, or their combinations in particular 3 and 5 and 6, or 4 and 5 and 6.).

In a still further aspect, the present invention provides a recombinant vector such as a recombinant virus. For example, the invention provides a modified recombinant virus; for instance, a recombinant virus modifed by having virus-encoded genetic functions inactivated (e.g., disrupted or deleted), such as a recombinant virus modified by having nonessential virus-encoded genetic functions inactivated therein, so that the virus has attenuated virulence; and, wherein the modified recombinant virus further contains DNA from a heterologous source in a the virus genome, such as in a nonessential region of the virus genome. The DNA can code for a PRRSV epitope of interest or can be PRRSV gene(s) or portion(s) thereof such as any or all of PRRSV ORF 2, ORF 3, ORF 4, ORF 5, ORF 6, ORF 7, e.g., PRRSV ORFs 2 and 3, or 3 and 4, or 3 and 5, or 3 and 5 and 6, or 4 and 5 and 6, or 3 and 4 and 5 and 6 expressed by any one or more of these ORFs or combinations of ORFs. In particular, the genetic functions can be inactivated by deleting an open reading frame encoding a virulence factor or by utilizing naturally host-restricted viruses and/or by utilizing attenuated and naturally-host restricted viruses. The virus used according to the present invention is advantageously a poxvirus, such as a vaccinia virus or preferably an avipox virus, e.g., a fowlpox virus or more preferably a canarypox virus. Advantageously, the open reading frame, with respect to vaccinia, is selected from the group consisting of J2R, B13R+B14R, A26L, A56R, C7L−K1L, I4L, combinations thereof (by the terminology reported in Goebel et al., 1990); and advantageously, the combination thereof comprising J2R, B13R+B14R, A26L, A56R, C7L−K1L, and I4L. In this respect, the open reading frame comprises a thymidine kinase gene, a hemorrhagic region, an A type inclusion body region, a hemagglutinin gene, a host range gene region or a large subunit, ribonucleotide reductase; or, the combination thereof. A suitable modified Copenhagen strain of vaccinia virus is identified as NYVAC (Tartaglia et al., 1992), or a vaccinia virus from which has been deleted J2R, B13R+B14R, A26L, A56R, C7L-K11 and 14L or a thymidine kinase gene, a hemorrhagic region, an A type inclusion body region, a hemagglutinin gene, a host range region, and a large subunit, ribonucleotide reductase (See also U.S. Pat. Nos. 5,364,773, 5,494,807 and 5,762,938 with respect to NYVAC and vectors having additional deletions or inactivations than those of NYVAC that are useful in the practice of this invention).

Preferably, the poxvirus vector is an ALVAC or, a canarypox virus which was attenuated, for instance, through more than 200 serial passages on chick embryo fibroblasts (Rentschler vaccine strain), a master seed therefrom was subjected to four successive plaque purifications under agar from which a plaque clone was amplified through five additional passages (See also U.S. Pat. Nos. 5,756,103 and 5,766,599 with respect to ALVAC and TROVAC (an attenuated fowlpox virus useful in the practice of this invention); and U.S. Pat. Nos. 6,004,777 and 5,990,091 and U.S. applications Ser. Nos. 60/151,564, 60/138,352 and 60/138,478, respectively filed Aug. 31, 1999, Jun. 10, 1999, and Jun. 10, 1999, with respect to vectors, such as vectors having enhanced expression and/or vectors useful with respect to porcine hosts (for instance, vectors useful with porcine hosts can include pig herpes viruses, including Aujeszky's disease virus, an adenovirus including a porcine adenovirus, a poxvirus, including a vaccinia virus, an avipox virus, a canarypox virus, a raccoonpox virus and a swinepox virus; see, e.g, U.S. Pat. Nos. 6,033,904, 5,869,312, and 5,382,425 with respect to swinepox), that also can be used in the practice of this invention, as well as with respect to terms used and teachings herein such as “immunogenic composition”, “immunological composition”, “vaccine”, and “epitope of interest”, and dosages, routes of administration, formulations, adjuvants, and uses for recombinant viruses and expression products therefrom). It is desirable that the recombinant vector or recombinant virus have expression without productive replication in the host.

As to epitopes of interest, reference is made to Kendrew, THE ENCYCLOPEDIA OF MOLECULAR BIOLOGY (Blackwell Science Ltd., 1995) and Sambrook, Fritsch and Maniatis, Molecular Cloning, A Laboratory Manual, 2nd Ed., Cold Spring Harbor Laboratory Press, 1982. An epitope of interest is an immunologically relevant region of an immunogen or immunologically active fragment thereof, e.g., from a pathogen or toxin of veterinary or human interest, e.g., PRRSV. One skilled in the art can determine an epitope or immunodominant region of a peptide or polypeptide and ergo the coding DNA therefor from the knowledge of the amino acid and corresponding DNA sequences of the peptide or polypeptide, as well as from the nature of particular amino acids (e.g., size, charge, etc.) and the codon dictionary, without undue experimentation.

The DNA sequence preferably encodes at least regions of the peptide that generate an antibody response or a T cell response. One method to determine T and B cell epitopes involves epitope mapping. The protein of interest is synthetized in short overlapping peptides (PEPSCAN). The individual peptides are then tested for their ability to bind to an antibody elicited by the native protein or to induce T cell or B cell activation. Janis Kuby, Immunology, (1992) pp.79-80.

Another method for determining an epitope of interest is to choose the regions of the protein that are hydrophilic. Hydrophilic residues are often on the surface of the protein and are therefore often the regions of the protein which are accessible to the antibody. Janis Kuby, Immunology, (1992) p. 81

Still another method for choosing an epitope of interest which can generate a T cell response is to identify from the protein sequence potential HLA anchor binding motifs which are peptide sequences which are known to be likely to bind to the MHC molecule.

The peptide which is a putative epitope of interest, to generate a T cell response, should be presented in a MHC complex. The peptide preferably contains appropriate anchor motifs for binding to the MHC molecules, and should bind with high enough affinity to generate an immune response.

Some guidelines in determining whether a protein is an epitopes of interest which will stimulate a T cell response, include: Peptide length—the peptide should be at least 8 or 9 amino acids long to fit into the MHC class I complex and at least 13-25 amino acids long to fit into a class II MHC complex. This length is a minimum for the peptide to bind to the MHC complex. It is preferred for the peptides to be longer than these lengths because cells may cut the expressed peptides. The peptide should contain an appropriate anchor motif which will enable it to bind to the various class I or class II molecules with high enough specificity to generate an immune response (See Bocchia, M. et al, Specific Binding of Leukemia Oncogene Fusion Protein Peptides to HLA Class I Molecules, Blood 85:2680-2684; Englehard, VH, Structure of peptides associated with class I and class II MHC molecules Ann. Rev. Immunol. 12:181 (1994)). This can be done, without undue experimentation, by comparing the sequence of the protein of interest with published structures of peptides associated with the MHC molecules.

Further, the skilled artisan can ascertain an epitope of interest by comparing the protein sequence with sequences listed in the protein data base.

Even further, another method is simply to generate or express portions of a protein of interest, generate monoclonal antibodies to those portions of the protein of interest, and then ascertain whether those antibodies inhibit growth in vitro of the pathogen from which the from which the protein was derived. The skilled artisan can use the other guidelines set forth in this disclosure and in the art for generating or expressing portions of a protein of interest for analysis as to whether antibodies thereto inhibit growth in vitro.

As to “immunogenic composition”, “immunological composition” and “vaccine”, an immunological composition containing the vector (or an expression product thereof) elicits an immunological response—local or systemic. The response can, but need not be protective. An immunogenic composition containing the inventive recombinant or vector (or an expression product thereof) likewise elicits a local or systemic immunological response which can, but need not be, protective. A vaccine composition elicits a local or systemic protective response. Accordingly, the terms “immunological composition” and “immunogenic composition” include a “vaccine composition” (as the two former terms can be protective compositions). The invention comprehends immunological, immunogenic or vaccine compositions.

With respect to dosages, routes of administration, formulations, adjuvants, and uses for recombinant viruses and expression products therefrom, compositions of the invention may be used for parenteral or mucosal administration, preferably by intradermal, subcutaneous or intramuscular routes. When mucosal administration is used, it is possible to use oral, ocular or nasal routes. The invention in yet a further aspect relates to the product of expression of the inventive recombinant or vector, e.g., virus, for instance, recombinant poxvirus, and uses therefor, such as to form an immunological or vaccine compositions for treatment, prevention, diagnosis or testing; and, to DNA from the recombinant or inventive virus, e.g., poxvirus, which is useful in constructing DNA probes and PCR primers.

In one aspect, the present invention provides a recombinant vector, e.g., virus such as a recombinant poxvirus containing therein a DNA sequence from PRRSV, e.g., in the virus (such as poxvirus) genome, advantageously a non-essential region of the virus, e.g., poxvirus genome. The poxvirus can be a vaccinia virus such as a NYVAC or NYVAC-based virus; and, the poxvirus is advantageously an avipox virus, such as fowlpox virus, especially an attenuated fowlpox virus, e.g., TROVAC, or a canarypox virus, preferably an attenuated canarypox virus, such as ALVAC.

According to the present invention, the recombinant vector, e.g., virus such as poxvirus, expresses gene products of the foreign PRRSV gene(s) or nucleic acid molecule(s). Specific ORF(s) of PRRSV or specific nucleic acid molecules encoding epitope(s) from specific PRRSV ORF(s) is/are inserted into the recombinant vector e.g., virus such as poxvirus vector, and the resulting vector, e.g., recombinant virus such as poxvirus, is used to infect an animal or express the product(s) in vitro for administration to the animal. Expression in the animal of PRRSV gene products results in an immune response in the animal to PRRSV. Thus, the recombinant vector, e.g., virus such as recombinant poxvirus of the present invention may be used in an immunological composition or vaccine to provide a means to induce an immune response.

The administration procedure for a recombinant vector, e.g., recombinant virus such as recombinant poxvirus-PRRSV or expression product thereof, as well as for compositions of the invention such as immunological or vaccine compositions or therapeutic compositions (e.g., compositions containing the recombinant vector or recombinant virus such as poxvirus or the expression product therefrom), can be via a parenteral route (intradermal, intramuscular or subcutaneous). Such an administration enables a systemic immune response, or humoral or cell-mediated responses.

The vector or recombinant virus-PRRSV, e.g., poxvirus-PRRSV, or expression product thereof, or composition containing such an expression product and/or vector or virus, can be administered to pigs of any age or sex; for instance, to elicit an immunological response against PRRSV, e.g., to thereby prevent PRRSV and/or other pathologic sequelae associated with PRRSV. Advantageously, the vector or recombinant virus-PRRSV, e.g., poxvirus-PRRSV, or expression product thereof, or composition containing such an expression product and/or vector or virus, is administered to a piglet or very young pig, including a newborn and/or to a pregnant sow to confer active immunity during gestation and/or passive immunity to the newborn through maternal antibodies. In a preferred embodiment, the invention provides for inoculation of a female pig (e.g., sow, gilt) with a composition comprising an immunogen from PRRSV or an epitope of interest from such an immunogen, e.g., an immunogen from PRRSV ORF 2, ORF 3, ORF 4, ORF 5, ORF 6, and/or ORF 7, for instance, an immunogen from PRRSV ORFs 3 and 5, or 5 and 6, or 4 and 5, or their combinations in particular 3 and 5 and 6, or 4 and 5 and 6 and/or an epitope of interest expressed by any one or more of these ORFs or combinations of ORFs, and/or with a vector expressing such an immunogen or epitope of interest. The inoculation can be prior to breeding; and/or prior to serving; and/or during gestation (or pregnancy), and/or prior to the perinatal period or farrowing; and/or repeatedly over a lifetime. Advantageously, at least one inoculation is done before serving. It is also advantageously followed by an inoculation to be performed during gestation, e.g., at about mid-gestation (at about 6-8 weeks of gestation) and/or at the end of gestation (or at about 11-13 weeks of gestation). Thus, an advantageous regimen is an inoculation before serving and a booster inoculation during gestation. Thereafter, there can be reinoculation before each serving and/or during gestation at about mid-gestation (at about 6-8 weeks of gestation) and/or at the end of gestation (or at about 11-13 weeks of gestation). Preferably, reinoculation can be during gestation only. In another preferred embodiment, piglets, such as piglets from vaccinated females (e.g., inoculated as herein discussed), are inoculated within the first weeks of life, e.g., inoculation at one and/or two and/or three and/or four and/or five weeks of life. More preferably, piglets are first inoculated within the first week of life or within the third week of life (e.g., at the time of weaning). Even more advantageous, such piglets are then boosted two (2) to four (4) weeks later (after being first inoculated). Thus, both offspring, as well as female pig (e.g., sow, gilt) can be administered compositions of the invention and/or can be the subject of performance of methods of the invention. Inoculations can be in the doses as herein described. With respect to the administration of poxvirus or virus compositions and maternal immunity, reference is made to U.S. Pat. No. 5,338,683.

The inventive recombinant vector or virus-PRRSV (e.g., poxvirus-PRRSV recombinants) immunological or vaccine compositions or therapeutic compositions, can be prepared in accordance with standard techniques well known to those skilled in the pharmaceutical or veterinary art. Such compositions can be administered in dosages and by techniques well known to those skilled in the veterinary arts taking into consideration such factors as the age, sex, weight, and the route of administration. The compositions can be administered alone, or can be co-administered or sequentially administered with compositions, e.g., with “other” immunological composition, or attenuated, inactivated, recombinant vaccine or therapeutic compositions thereby providing multivalent or “cocktail” or combination compositions of the invention and methods employing them. The composition may contain combinations of the PRRSV component (e.g., recombinant vector such as a plasmid or virus or poxvirus expressing a PRRSV immunogen or epitope of interest and/or PRRSV immunogen or epitope of interest) and one or more unrelated porcine pathogen vaccines (e.g., epitope(s) of interest, immunogen(s) and/or recombinant vector or virus such as a recombinant virus, e.g., recombinant poxvirus expressing such epitope(s) or immunogen(s)) such as one or more immunogen or epitope of interest from one or more porcine bacterial and/or viral pathogen(s), e.g., an epitope of interest or immunogen from one or more of: porcine circovirus, porcine parvovirus, porcine influenza virus, pseudorabies virus, E. coli, Erysipelothrix rhusiopathiae, Mycoplasma hyopneumoniae, Pasteurella multocida, Bordetella bronchiseptica, Actinobacillus pneumoniae, hog cholera virus, and the like. Again, the ingredients and manner (sequential or co-administration) of administration, as well as dosages can be determined taking into consideration such factors as the age, sex, weight, and, the route of administration. In this regard, reference is made to U.S. Pat. No. 5,843,456, incorporated herein by reference, and directed to rabies compositions and combination compositions and uses thereof; see also other documents cited herein and documents cited or referenced in herein cited documents, including U.S. application Ser. No. 60/151,564; and U.S. Pat. No. 6,217,883.

Examples of compositions of the invention include liquid preparations for mucosal administration, e.g., oral, nasal, ocular, etc., administration such as suspensions and, preparations for parenteral, subcutaneous, intradermal, intramuscular (e.g., injectable administration) such as sterile suspensions or emulsions. In such compositions the recombinant poxvirus or immunogens may be in admixture with a suitable carrier, diluent, or excipient such as sterile water, physiological saline, or the like. The compositions can also be lyophilized or frozen. The compositions can contain auxiliary substances such as wetting or emulsifying agents, pH buffering agents, adjuvants, preservatives, and the like, depending upon the route of administration and the preparation desired.

The compositions can contain at least one adjuvant compound chosen from the polymers of acrylic or methacrylic acid and the copolymers of maleic anhydride and alkenyl derivative.

The preferred adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 (incorporated herein by reference) which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol® (BF Goodrich, Ohio, USA) are particularly appropriate. They are cross-linked with allyl sucrose or with allyl pentaerythritol. Among them, there may be mentioned Carbopol® 974P, 934P and 971 P.

Among the copolymers of maleic anhydride and alkenyl derivative, the copolymers EMA® (Monsanto) which are copolymers of maleic anhydride and ethylene, linear or cross-linked, for example cross-linked with divinyl ether, are preferred. Reference may be made to J. Fields et al., Nature, 186:778-780, Jun. 4, 1960, incorporated herein by reference.

From the point of view of their structure, the polymers of acrylic or methacrylic acid and the copolymers EMA® are preferably formed of basic units of the following formula:

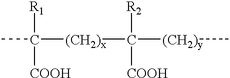

in which:

R₁ and R₂, which are identical or different, represent H or CH₃

x=0 or 1, preferably x=1

y=1 or 2,with x+y=2

For the copolymers EMA®, x=0 and y=2. For the carbomers, x=y=1.

The dissolution of these polymers in water leads to an acid solution which will be neutralized, preferably to physiological pH, in order to give the adjuvant solution into which the vaccine itself will be incorporated. The carboxyl groups of the polymer are then partly in COO⁻form.

Preferably, a solution of adjuvant according to the invention, especially of carbomer, is prepared in distilled water, preferably in the presence of sodium chloride, the solution obtained being at acidic pH. This stock solution is diluted by adding it to the desired quantity (for obtaining the desired final concentration), or a substantial part thereof, of water charged with NaCl, preferably physiological saline (NaCL 9 g/l) all at once in several portions with concomitant or subsequent neutralization (pH 7.3 to 7.4), preferably with NaOH. This solution at physiological pH will be used as it is for mixing with the vaccine, which may be especially stored in freeze-dried, liquid or frozen form.

The polymer concentration in the final vaccine composition will be 0.01% to 2% w/v, more particularly 0.06 to 1% w/v, preferably 0.1 to 0.6% w/v.

The compositions of the invention can also be formulated as oil in water or as water in oil in water emulsions, e.g. as in V. Ganne et al. Vaccine 1994, 12, 1190-1196.

Standard texts, such as “REMINGTON'S PHARMACEUTICAL SCIENCE”, 17th edition, 1985, incorporated herein by reference, may be consulted to prepare suitable preparations, without undue experimentation.

Compositions in forms for various administration routes are envisioned by the invention. And again, the effective dosage and route of administration are determined by known factors, such as age, sex, weight, and other screening procedures which are known and do not require undue experimentation. Dosages of each active agent can be as in herein cited documents (or documents referenced or cited in herein cited documents) and/or can range from one or a few to a few hundred or thousand micrograms, e.g., 1 μg to 1 mg, for a subunit immunogenic, immunological or vaccine composition.

Recombinant vectors can be administered in a suitable amount to obtain in vivo expression corresponding to the dosages described herein and/or in herein cited documents. For instance, suitable ranges for viral suspensions can be determined empirically. The viral vector or recombinant in the invention can be administered to a pig or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu, per dose, for example, per 2 ml dose. And, if more than one gene product is expressed by more than one recombinant, each recombinant can be administered in these amounts; or, each recombinant can be administered such that there is, in combination, a sum of recombinants comprising these amounts. In recombinant vector compositions employed in the invention, dosages can be as described in documents cited herein or as described herein or as in documents referenced or cited in herein cited documents. For instance, suitable quantities of each DNA in recombinant vector compositions can be 1 μg to 2 mg, preferably 50 μg to 1 mg. Documents cited herein (or documents cited or referenced in herein cited documents) regarding DNA vectors may be consulted by the skilled artisan to ascertain other suitable dosages for recombinant DNA vector compositions of the invention, without undue experimentation.

However, the dosage of the composition(s), concentration of components therein and timing of administering the composition(s), which elicit a suitable immunological response, can be determined by methods such as by antibody titrations of sera, e.g., by ELISA and/or seroneutralization assay analysis and/or by vaccination challenge evaluation in pig. Such determinations do not require undue experimentation from the knowledge of the skilled artisan, this disclosure and the documents cited herein. And, the time for sequential administrations can be likewise ascertained with methods ascertainable from this disclosure, and the knowledge in the art, without undue experimentation.

The PRRSV immunogen or epitope of interest can be obtained from PRRSV or can be obtained from in vitro recombinant expression of PRRSV gene(s) or portions thereof. Methods for making and/or using vectors (or recombinants) for expression and uses of expression products and products therefrom (such as antibodies) can be by or analogous to the methods disclosed in herein cited documents and documents referenced or cited in herein cited documents.

Suitable dosages can also be based upon the examples below.

The invention in a particular aspect is directed to recombinant poxviruses containing therein a DNA sequence from PRRSV, advantageously in a nonessential region of the poxvirus genome. The recombinant poxviruses express gene products of the foreign PRRSV gene. In particular, ORF 2, ORF 3, ORF 4, ORF 5, ORF 6, and ORF 7 genes encoding PRRSV proteins were isolated, characterized and inserted into ALVAC (canarypox vector) recombinants. Advantageously, the ALVAC canarypox vector contains and expresses PRRSV ORFs 3 and 5, or 5 and 6, or 4 and 5, or their combinations in particular 3 and 5 and 6, or 4 and 5 and 6. The molecular biology techniques used are the ones described by Sambrook et al. (1989).

The invention shall be further described by way of the following Examples, provided for illustration and not to be considered a limitation of the invention.

### EXAMPLES

Cell Lines and Virus Strains. The strain of PRRSV is P120-117B/13/Macro/1/27-01-93, which was isolated in Germany in 1991 from different organs of an infected piglet. See also ATCC VR-2332; U.S. Pat. No. 5,476,778; U.S. Pat. No. 5,620,691; WO 92/21375; I-1102, deposited Jun. 5, 1991 with the Institut Pasteur, Paris, France; Meulenberg J. et al., Virology, 192:62-72 (1993); Mardassi H. et al., Arch. Virol., 140:1405-1418 (1995); Den Boon et al., 1991; Godeny et al., 1993; Meulenberg et al. 1993a; WO 98/03658; PCT/FR97/01313; French application 96 09338; U.S. application Ser. No. 09/232,468; Murtaugh, 1995; and other documents cited herein.

The parental canarypox virus (Rentschler strain) is a vaccinal strain for canaries. The vaccine strain was obtained from a wild type isolate and attenuated through more than 200 serial passages on chick embryo fibroblasts. A master viral seed was subjected to four successive plaque purifications under agar and one plaque clone was amplified through five additional passages after which the stock virus was used as the parental virus in in vitro recombination tests. The plaque purified canarypox isolate is designated ALVAC. ALVAC was deposited Nov. 14, 1996 under the terms of the Budapest Treaty at the American Type Culture Collection, ATCC accession number VR-2547; see also documents cited herein.

The generation of poxvirus recombinants can involve different steps: (1) construction of an insertion plasmid containing sequences (“arms”) flanking the insertion locus within the poxvirus genome, and multiple cloning site (MCS) localized between the two flanking arms (e.g., see example 1); (2) construction of donor plasmids consisting of an insertion plasmid into the MCS of which a foreign gene expression cassette has been inserted (e.g. see examples 2 and 3); (3) in vitro recombination in cell culture between the arms of the donor plasmid and the genome of the parental poxvirus allowing the insertion of the foreign gene expression cassette into the appropriate locus of the poxvirus genome, and plaque purification of the recombinant virus (e.g. see example 10).

### Example 1

Construction of Canarypox Insertion Plasmid at C6 Locus

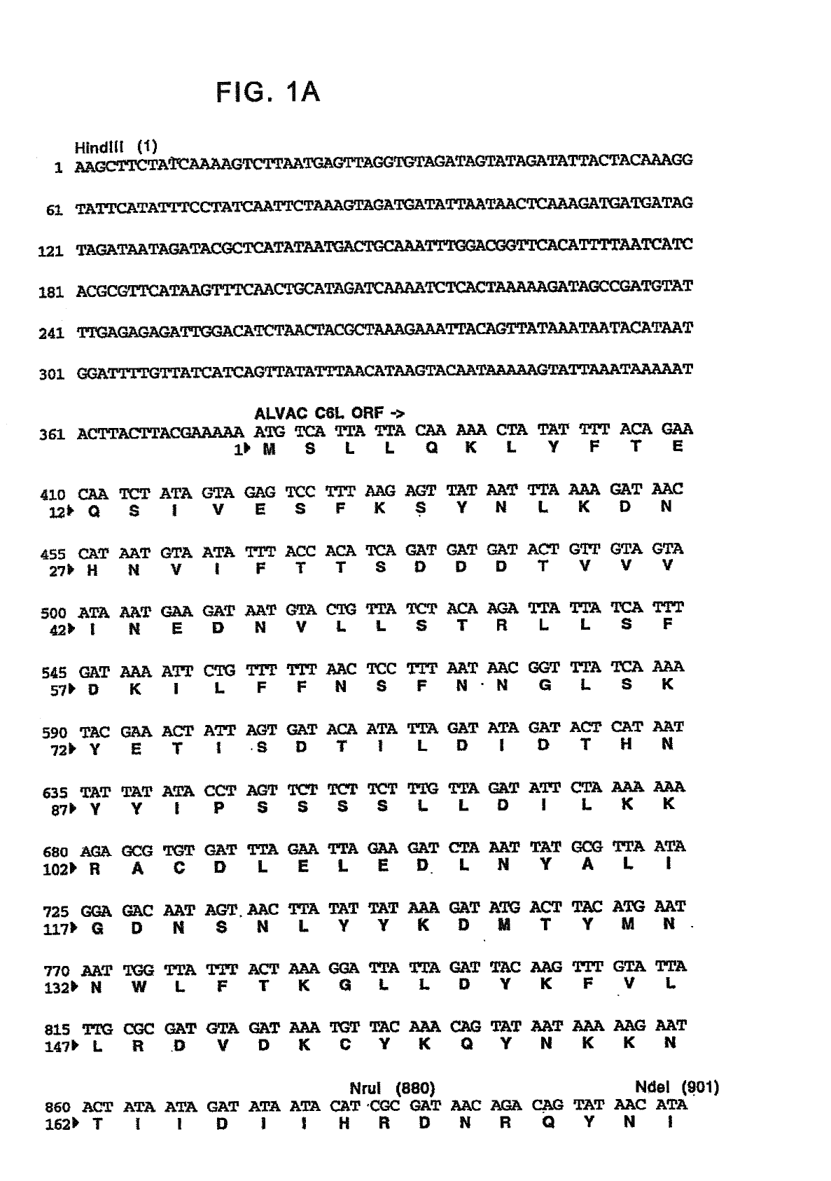

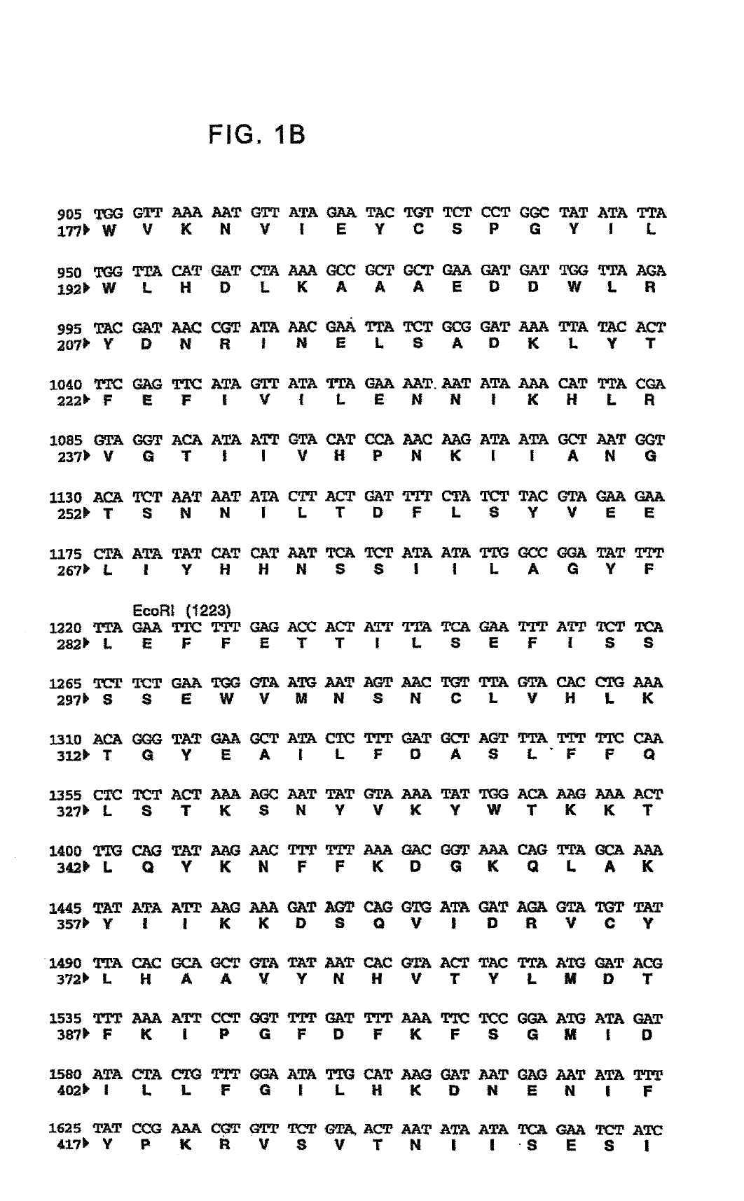

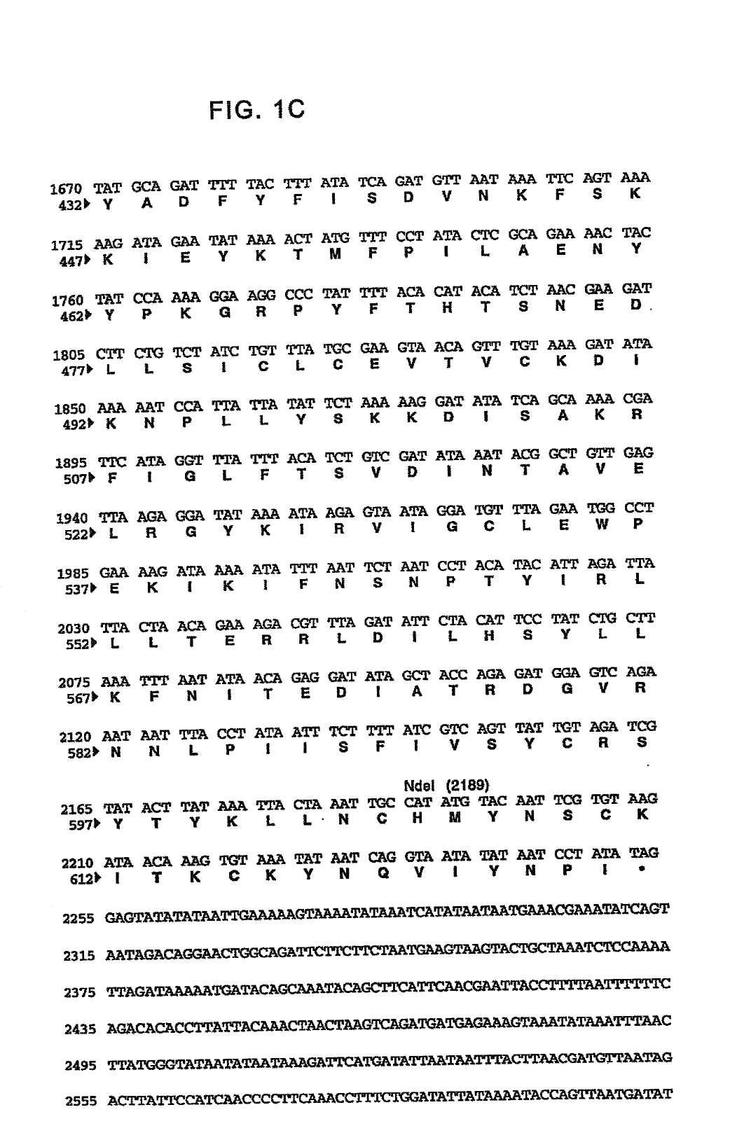

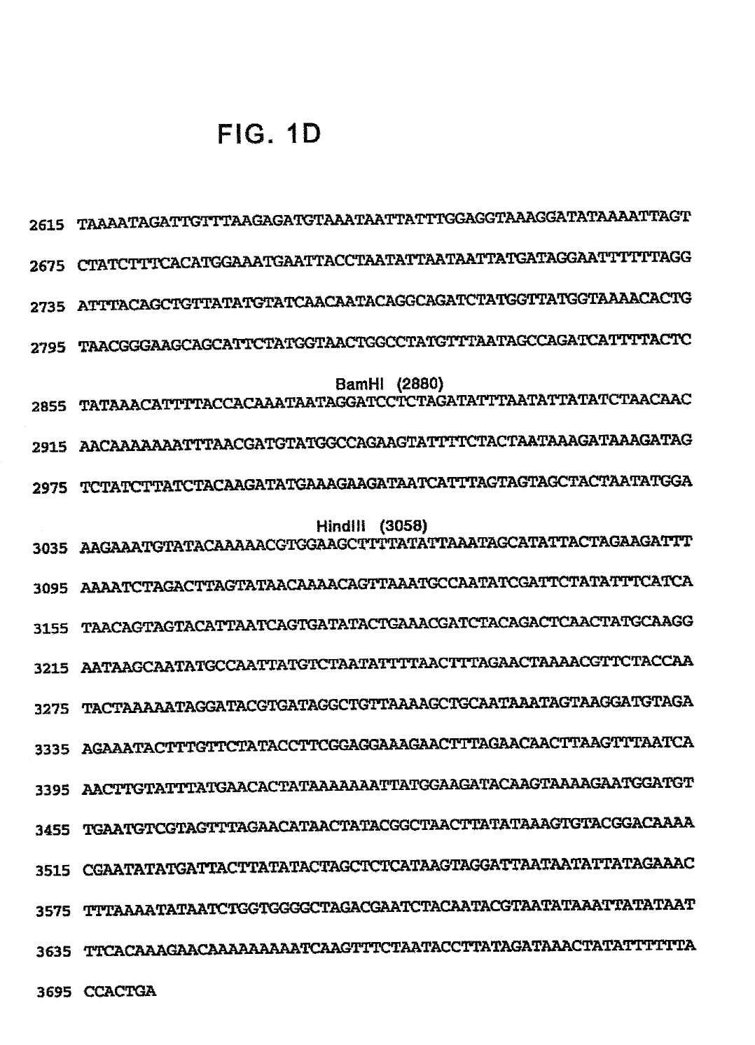

FIG. 1 (SEQ ID NO: 1) is the sequence of a 3.7 kb segment of canarypox DNA. Analysis of the sequence revealed an ORF designated C6L initiated at position 377 and terminated at position 2254. The following describes a C6 insertion plasmid constructed by deleting the C6 ORF and replacing it with a multiple cloning site (MCS) flanked by transcriptional and translational termination signals. A 380 bp PCR fragment is amplified from genomic canarypox DNA using oligonucleotide primers C6A1 (SEQ ID NO: 2) and C6B1 (SEQ ID NO: 3). A 1155 bp PCR fragment is amplified from genomic canarypox DNA using oligonucleotide primers C6C1 (SEQ ID NO: 4) and C6D1 (SEQ ID NO: 5). The 380 bp and 1155 bp fragments are fused together by adding them together as template and amplifying a 1613 bp PCR fragment using oligonucleotide primers C6A1 (SEQ ID NO: 2) and C6D1 (SEQ ID NO: 5). This fragment is digested with SacI and KpnI, and ligated into pBluescript SK+ digested with SacI/KpnI. The resulting plasmid, pC6L is confirmed by DNA sequence analysis. It consists of 370 bp of canarypox DNA upstream of C6 (“C6 left arm”), vaccinia early termination signal, translation stop codons in six reading frames, an MCS containing SmaI, PstI, XhoI and EcoRI sites, vaccinia early termination signal, translation stop codons in six reading frames and 1156 bp of downstream canary pox sequence (“C6 right arm”).

Plasmid pJP099 is derived from pC6L by ligating a cassette containing the vaccinia H6 promoter (described in Taylor et al. (1988c), Guo et al. (1989), and Perkus et al. (1989)) coupled to a foreign gene into the SmaIEcoRI sites of pC6L. This plasmid pJPO99 contains a unique EcoRV site and a unique NruI site located at the 3′ end of the H6 promoter, and a unique SalI and PspAI site located between the STOP codon of the foreign gene and the C6 left arm. The ˜4.5 kb EcoRVI/SalI or EcoRV/PspAI fragment from pJP099 contains therefore the plasmid sequence (pBluescript SK+; Stratagene, La Jolla, Calif., USA), the 2 C6 arms and the 5′ end of the H6 promoter until the EcoRV. Plasmid pJP105 is a derivative of pJP099 containing a different foreign gene inserted into pC6L. The ˜4.5 kb EcoRV/SalI fragment from pJP105 is identical to that of pJP099 described above.

Sequences of the Primers

| | |
|---|---|
| Primer C6A1 | |
| ATCATCGAGCTCGCGGCCGCCTATCAAAAGTCTTAATGAGTT | (SEQ ID NO:2) |
| Primer C6B1 | |
| GAATTCCTCGAGCTGCAGCCCGGGTTTTTATAGCTAATTAGTCATTTTTTCGTAAGTA | (SEQ ID NO:3) |
| AGTATTTTTATTTAA | |
| Primer C6C1 | |
| CCCGGGCTGCAGCTCGAGGAATTCTTTTTATTGATTAACTAGTCAAATGAGTATATA | (SEQ ID NO:4) |
| TAATTGAAAAAGTAA | |
| Primer C6D1 | |
| GATGATGGTACCTTCATAAATACAAGTTTGATTAAACTTAAGTTG | (SEQ ID NO:5) |

### Example 2

Production of PRRSV and Extraction of Viral RNA

The PRRSV strain P120-117B/13/Macro/1/27-01-93 is amplified in MA104 cells with DMEM medium supplemented with 5% fetal calf serum. Infected cells are harvested after 4 days of incubation at 37° C. The cell debris are removed by centrifugation after 3 freezing thawing cycles.

Total RNA is extracted from the viral suspension according to the Micro-Scale Total RNA Separator Kit (Clontech Laboratories, Inc., Palo Alto, Calif. , U.S.A; Cat#K1044-1; for ORFs 4 to 7), or to the High Pure RNA Isolation Kit (Boehringer Mannheim Gmbh, Roche Molecular Biochemicals, Mannheim, Germany; ref 1828665; for ORFs 2 and 3). The RNA pellet is suspended in 20 μl DEPC-treated water.

### Example 3

Consturction of Alvac Donor Plasmid for PRRSV ORF 2

First strand cDNA synthesis is performed in 20 μl final volume consisting of 1 μl of viral RNA (see example 2) and 19 μl of RT-PCR MasterMix according to 1^{st} Strand cDNA synthesis Kit (Perkin Elmer, manufactured by Roche Molecular Systems Inc., Branchburg, N.J., U.S.A.; Cat#N808-0017). The MasterMix includes MgCl₂ (5 mM), PCR bufferII (1×), dNTPs (1 mM), Rnase inhibitor (1 U), Murine Leukemia Virus Reverse Transcriptase (2.5 U), and oligonucleotide PB613 (0.75 μM) used as a primer (SEQ ID NO: 6). Reaction mixture is successively incubated at 42° C. for 15 min, 99° C. for 5 min and 4° C. for 5 min. The single strand cDNA is subseq PCR-amplified in a 100 μl final volume consisting of the 20 μl RT-PCR reaction and 80 μl of PCR mix (10 μl of 10 × reaction buffer, 25 mM each dNTP, 2.5 U of cloned Pfu DNA Polymerase (ref#600154; Stratagene, La Jolla, Calif., USA)) including oligonucleotide primers PB612 (SEQ ID NO: 7) and PB613 (SEQ ID NO: 6). Thirty-five cycles of amplification (95° C. for 45 sec; 56° C. for 45 sec and 72° C. for 1 min) are performed. The 1534 bp fragment containing the PRRSV ORF 2 and ORF3 coding sequence is purified by Geneclean (GENECLEAN Kit; BIO 101, Vista, Calif., U.S.A.) and cloned into the pCRII plasmid (Invitrogen, Carlsbad, Calif., U.S.A.). The resulting plasmid is designated pPB356.

A consensus sequence for ORF 2 and 3 is derived from the insert sequence of three clones of pPB356. The consensus nucleotide sequence of ORF 2 differs from the reference sequence (PRRSV Lelystad strain, Genebank accession number M96262) at 5 positions (among 750 bp; 99% homology), 2 of which changing the amino acid sequence (amino acid 29: Ser in pPB356 and Pro in Lelystad; amino acid 122: Val in pPB356 and Ala in Lelystad). The consensus nucleotide sequence of ORF 3 differs from the reference sequence (PRRSV Lelystad strain, Genebank accession number M96262) at 6 positions (among 798 bp; 99% homology), 4 of which changing the amino acid sequence (amino acid 15: Val in pPB356 and Phe in Lelystad; amino acid 93: Pro in pPB356 and Ser in Lelystad; amino acid 102: Arg in pPB356 and Lys in Lelystad; amino acid 150: Gln in pPB356 and His in Lelystad). Clone pPB356.6A contains the consensus amino acid sequence for ORF 2 and ORF 3.

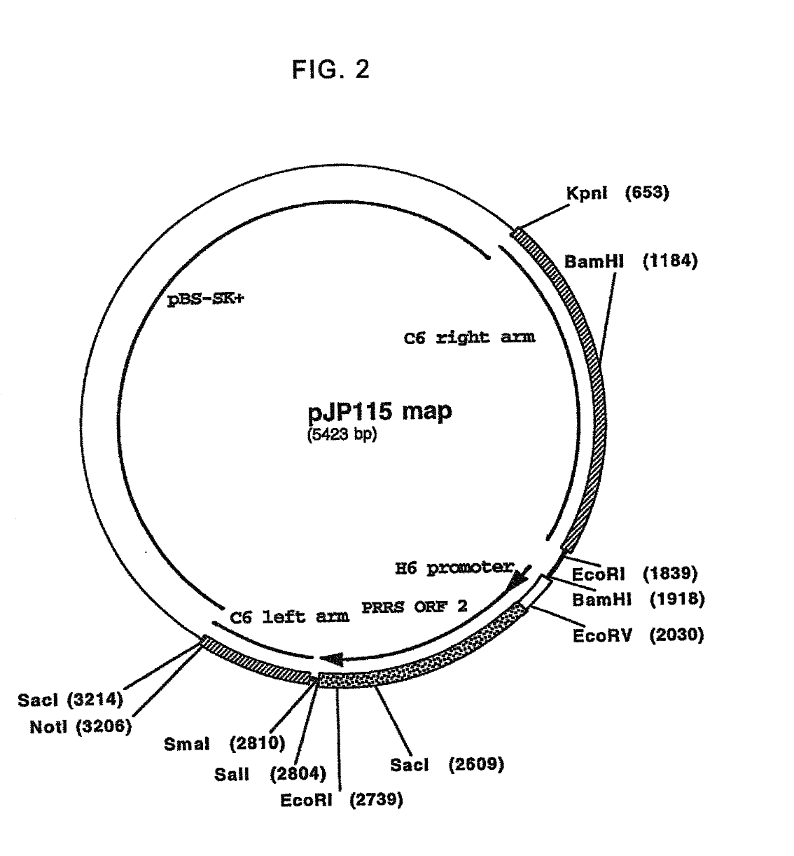

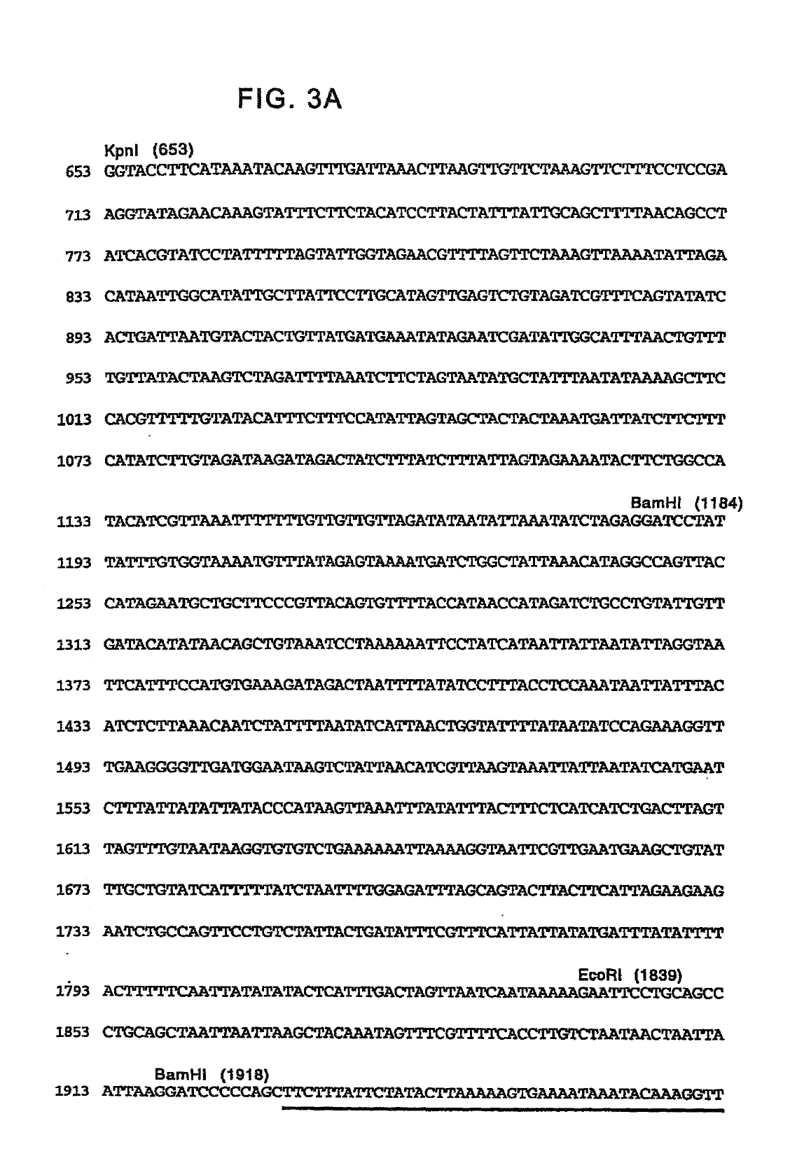

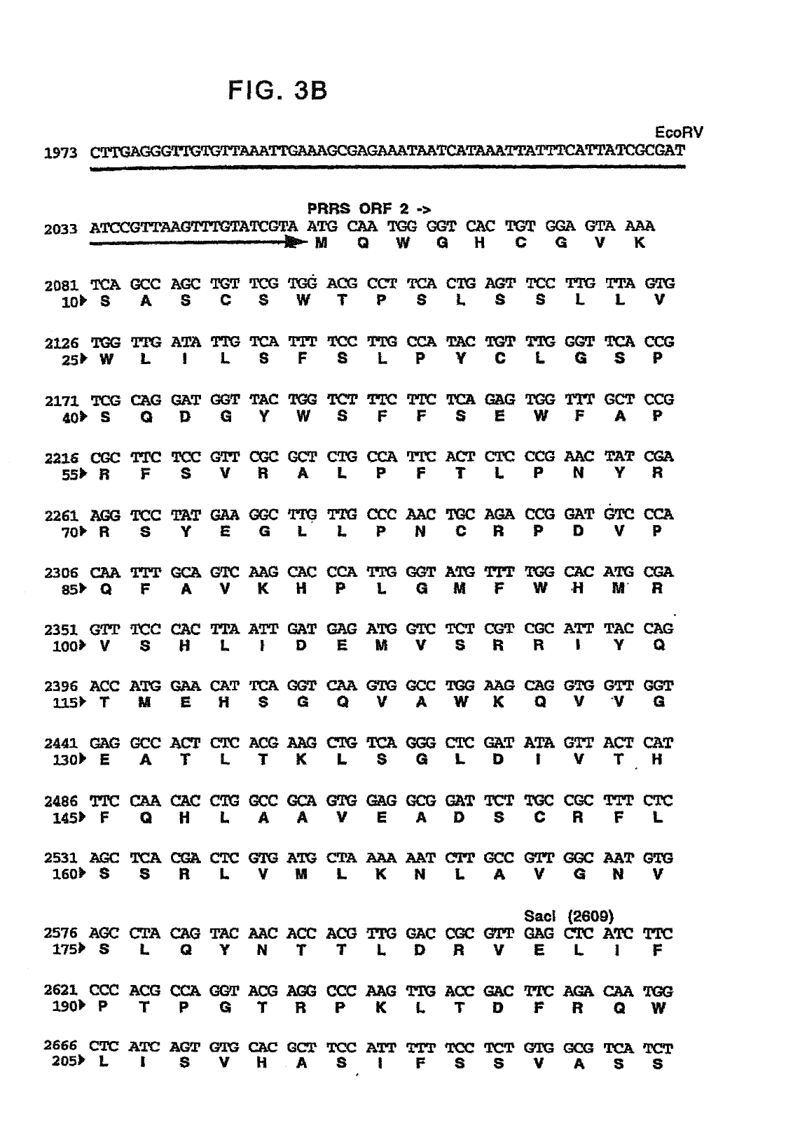

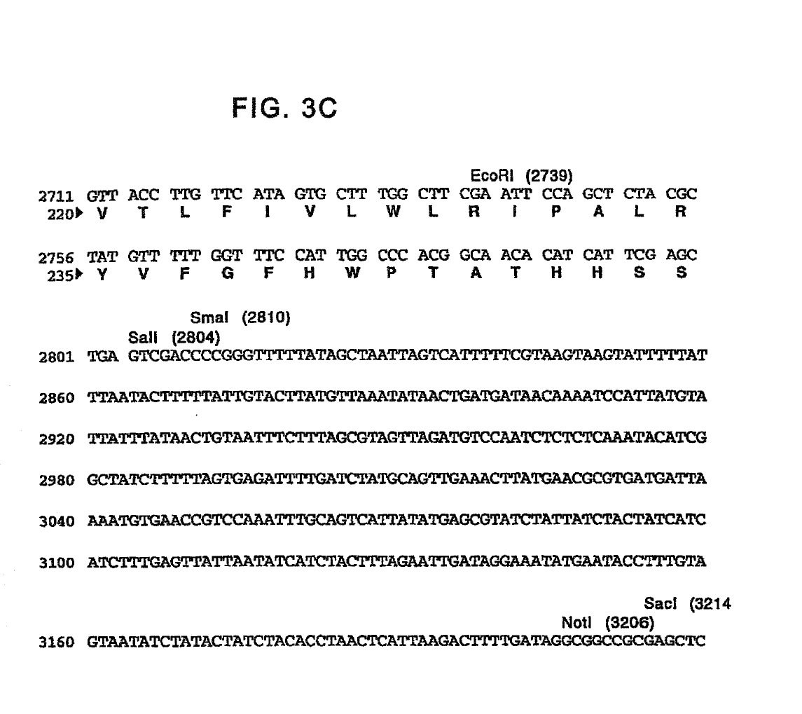

PRRSV ORF 2 is obtained by PCR using primers JP800 (SEQ ID NO: 8; contains the 3′ end of the H6 promoter (from the EcoRV) and the 5′ end of PRRS ORF2) and JP801 (SEQ ID NO: 9; it contains the 3′ end of PRRS ORF 2 and a SalI cloning site) on plasmid pPB356.6A to generate a ˜770 bp fragment designated PCR J1315. PCR J1315 is digested with EcoRV/SalI and cloned into a ˜4.5 kb EcoRV/SalI fragment from pJP105 (see example 1). The resulting plasmid is confirmed by sequence analysis and designated pJP115 (see the map in FIG. 2 and the sequence (SEQ ID NO: 10) in FIG. 3). This donor plasmid pJP115 (linearized with NotI) is used in an in vitro recombination (IVR) assay to generate ALVAC recombinant vCP1642 (see example 10).

Sequence of the Primers

Primer PB613 (SEQ ID NO:6) (downstream orf3)
TAG AAA AGG CAC GCA GAA AGC
Primer PB612 (SEQ ID NO:7) (upstream orf2)
CAG GTA GAG CTA GGT AAA CCC
Primer JP800 (SEQ ID NO:8)
CAT CAT CAT GAT ATC CGT TAA GTT TGT ATC GTA ATG CAA TGG GGT CAC TGT GG
Primer JP801 (SEQ ID NO:9)
TAC TAC TAC GTC GAC TCA GCT CGA ATG ATG TGT TGC

### Example 4

Construction of Alvac Donor Plasmid for PRRSV ORF 3

The PRRSV ORF 3 sequence from the clone pPB356.6A (see example 3) contains a T5NT at position 334-340, which is known to be an early transcription termination signal in poxvirus, and therefore, needs to be silently mutated. ORF 3 is amplified by PCR using primers JP804 (SEQ ID NO: 11; contains the 3′ end of the H6 promoter (from the EcoRV site) and the 5′ end of PRRS ORF3) and JP805 (SEQ ID NO: 12; contains the 3′ end of PRRS ORF3 and a SalI cloning site) on plasmid pPB356.6A to generate a ˜820 bp fragment designated PCR J1317A. PCR J1317A is digested with EcoRV/SalI and cloned into a ˜4.5 kb EcoRV/SalI fragment from pJP105 (see example 1). The ORF 3 sequence of two clones of the resulting plasmid, designated 8S20 and 8S19 is sequenced. ORF 3 correct sequence is found before (5′) and after (3′) the T5NT signal in clone 8S19 and 8S20, respectively.

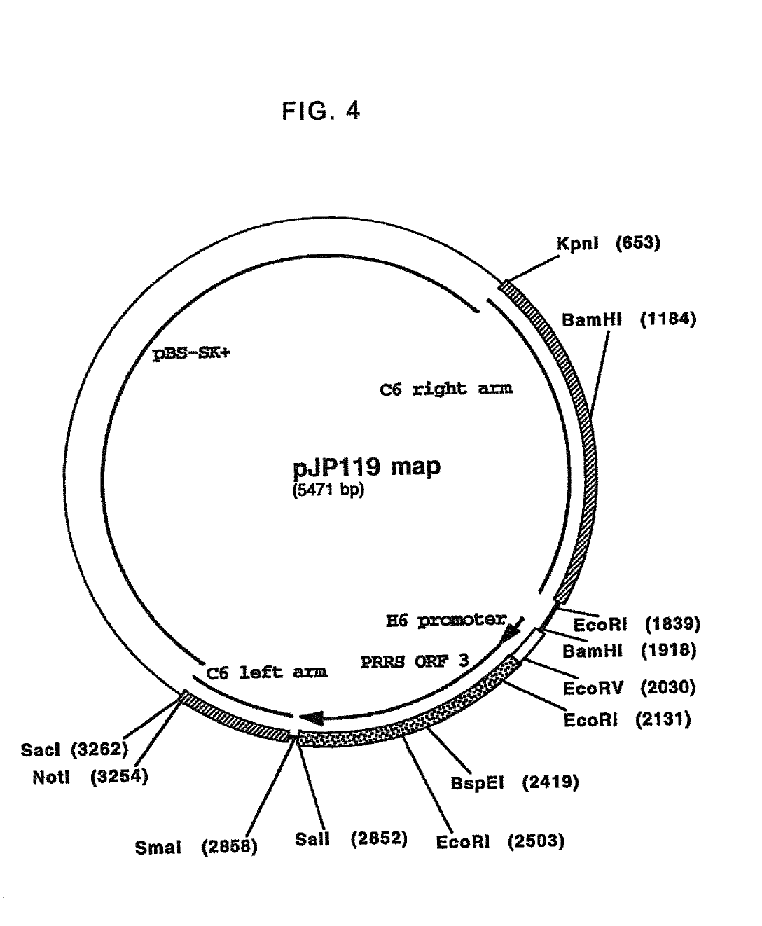

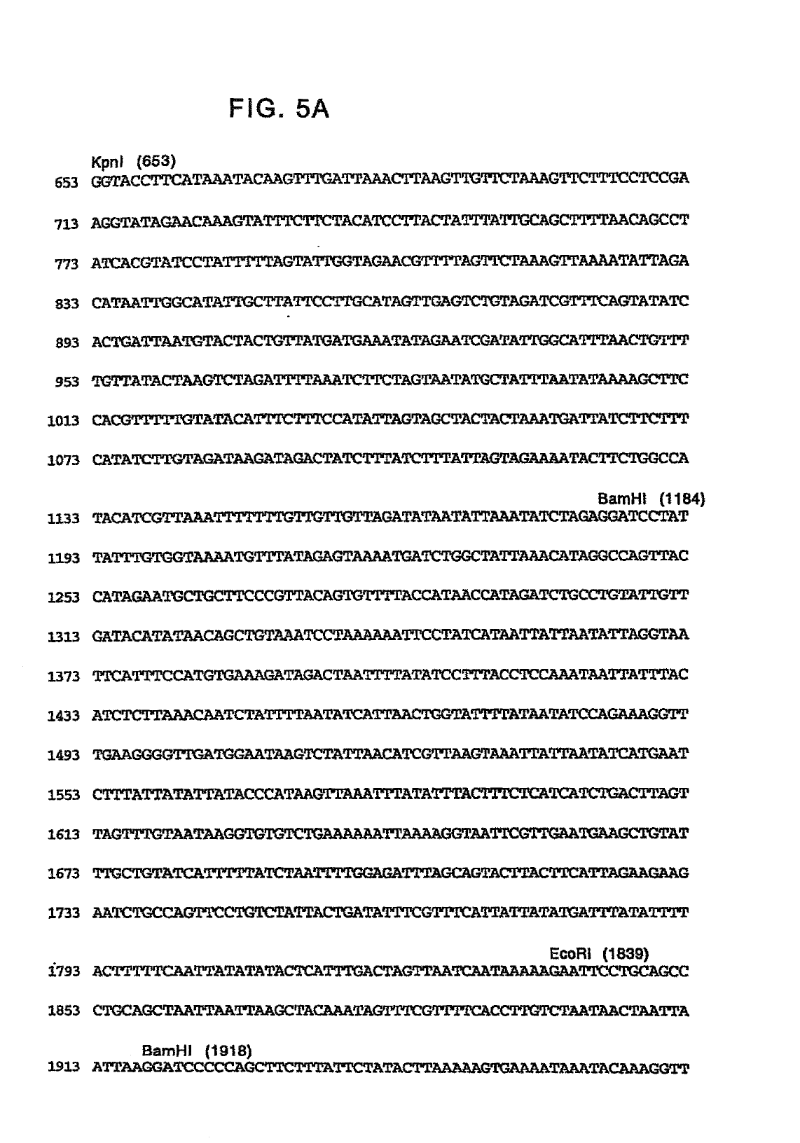

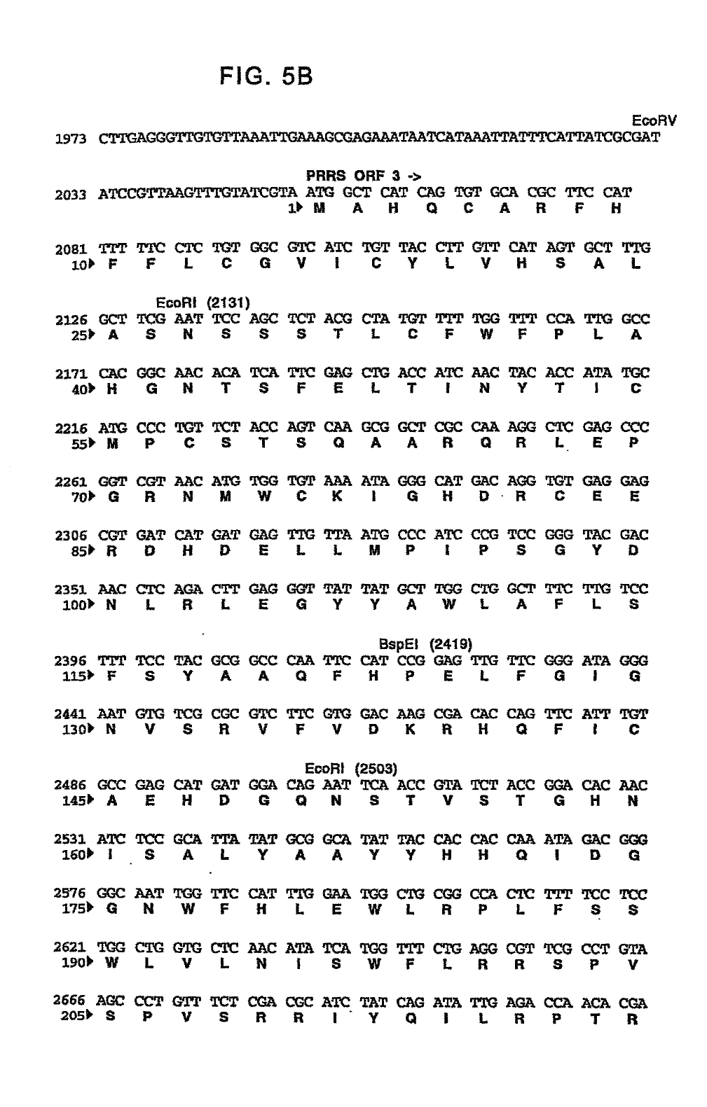

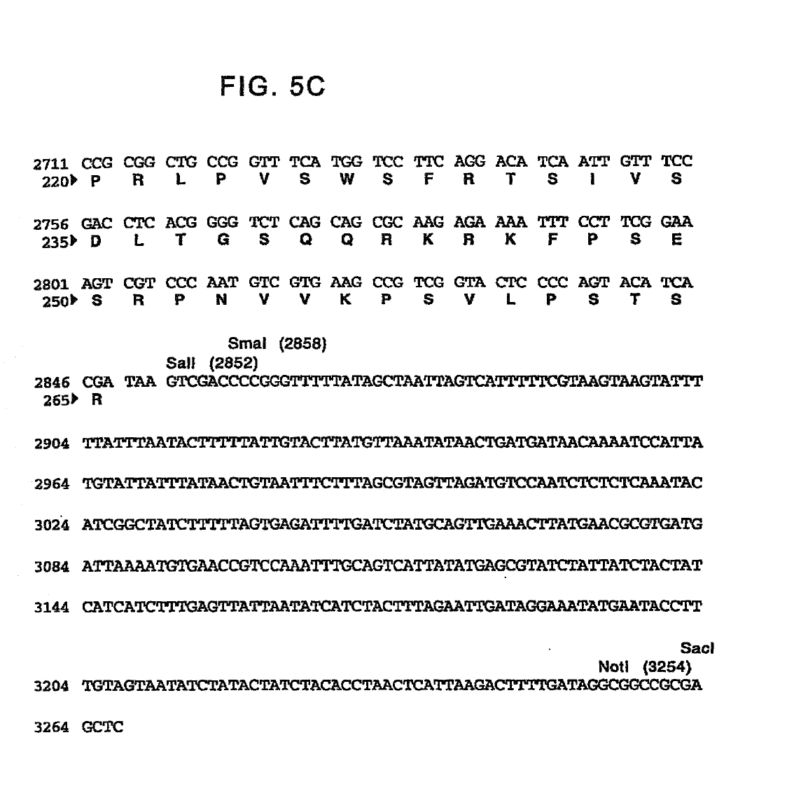

In order to mutate the T5NT, PCR J1319 is generated using primers JP804 (SEQ ID NO: 11) and JP821 (SEQ ID NO: 13; contains sequence starting downstream of the BspEI site and continuing through the T5NT change) on plasmid 8S19. PCR J1319 is digested with EcorV/BspEI and ligated into EcoRV/BspEI digested 8S20. The resulting plasmid, designated pJP 119, is confirmed by sequence analysis (see the map in FIG. 4 and the sequence (SEQ ID NO: 14) in FIG. 5). This donor plasmid pJP119 (linearized with NotI) is used in an in vitro recombination (IVR) assay to generate ALVAC recombinant vCP 1643 (see example 10).

Sequence of the primers

Primer JP804 (SEQ ID NO:11)
CAT CAT CAT GAT ATC CGT TAA GTT TGT ATC GTA ATG GCT CAT CAG TGT GCA CG
Primer JP805 (SEQ ID NO:12)
TAC TAC TAC GTC GAC TTA TCG TGA TGT ACT GGG GAG
Primer JP821 (SEQ ID NO:13)
TAT CCC GAA CAA CTC CGG ATG GAA TTG GGC CGC GTA GGA AAA GGA CAA
GAA AGC CAG CCA AGC

### Example 5

CONSTRUCTION OF ALVAC DONOR PLASMID FOR PRRSV ORF 4

First strand cDNA synthesis is performed in 20 μl final volume consisting of 1 μl of viral RNA (see example 2) and 19 μl of RT-PCR MasterMix according to 1^{st} Strand cDNA synthesis Kit (Perkin Elmer, manufactured by Roche Molecular Systems Inc., Branchburg, N.J., U.S.A.; Cat#N808-0017). The MasterMix includes MgCl₂ (5 mM), PCR bufferII (1×), dNTPs (1 mM), Rnase inhibitor (1 U), Murine Leukemia Virus Reverse Transcriptase (2.5 U), and oligonucleotide PB472 (0.75 μM) used as a primer (SEQ ID NO: 15). Reaction mixture is successively incubated at 42° C. for 15 min, 99° C. for 5 min and 4° C. for 5 min. The single strand cDNA is subsequently PCR-amplified in a 100 μl final volume consisting of the 20 μl RT-PCR reaction and 80 μl of PCR MasterMix (2 mM MgCl₂, PCR bufferII 1× and 2.5 U Ampli Taq® DNA Polymerase) including oligonucleotide primers PB471 (SEQ ID NO: 16) and PB472 (SEQ ID NO: 15). After a first 2 min. incubation at 95° C., 35 cycles of amplification (96° C. for 45 sec; 56° C. for 72° C. for 1.5 min) are performed. The PCR fragment containing the PRRSV ORF 4 coding sequence is purified by Geneclean (GENECLEAN Kit; BIO101 Vista, Calif., U.S.A.) and subsequently digested with SalI and BamHI to generate a SalI-BamHI fragment of 595bp. This fragment is cloned into the vector pVR1012 (VICAL Inc., San Diego, Calif., U.S.A.) digested with SalI and BamHI to generate plasmid pPB272. The ORF 4 present in three independent clones are sequenced in their entirety and a consensus sequence is established and compared to the sequence of reference (PRRSV Lelystad strain, Genebank accession number M96262). Four base pair mutations (in the 552 bp sequence of ORF 4; 99% homology) are found, but only one induces an amino acid change (amino acid 8: Phe and Leu in Lelystad and pPB272, respectively).

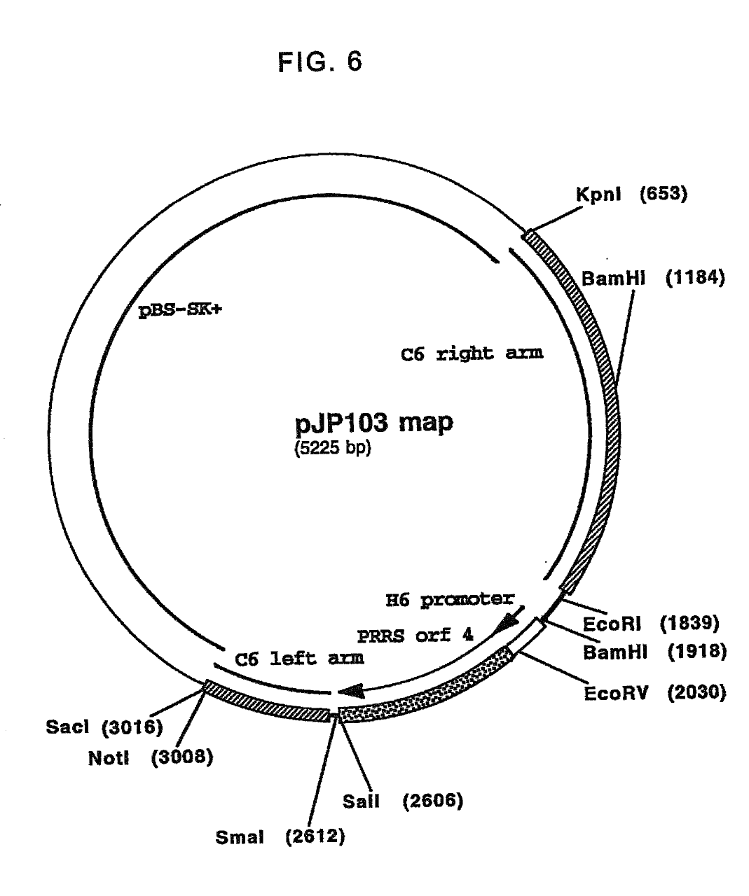

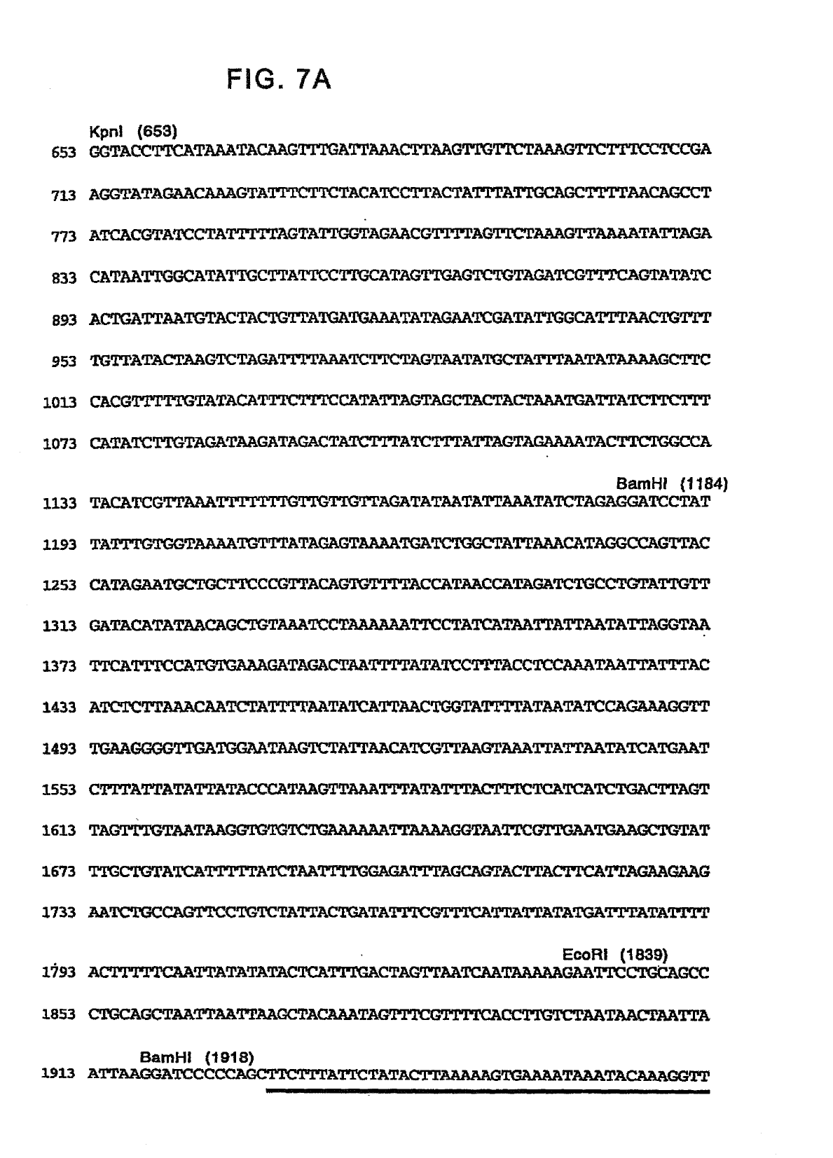

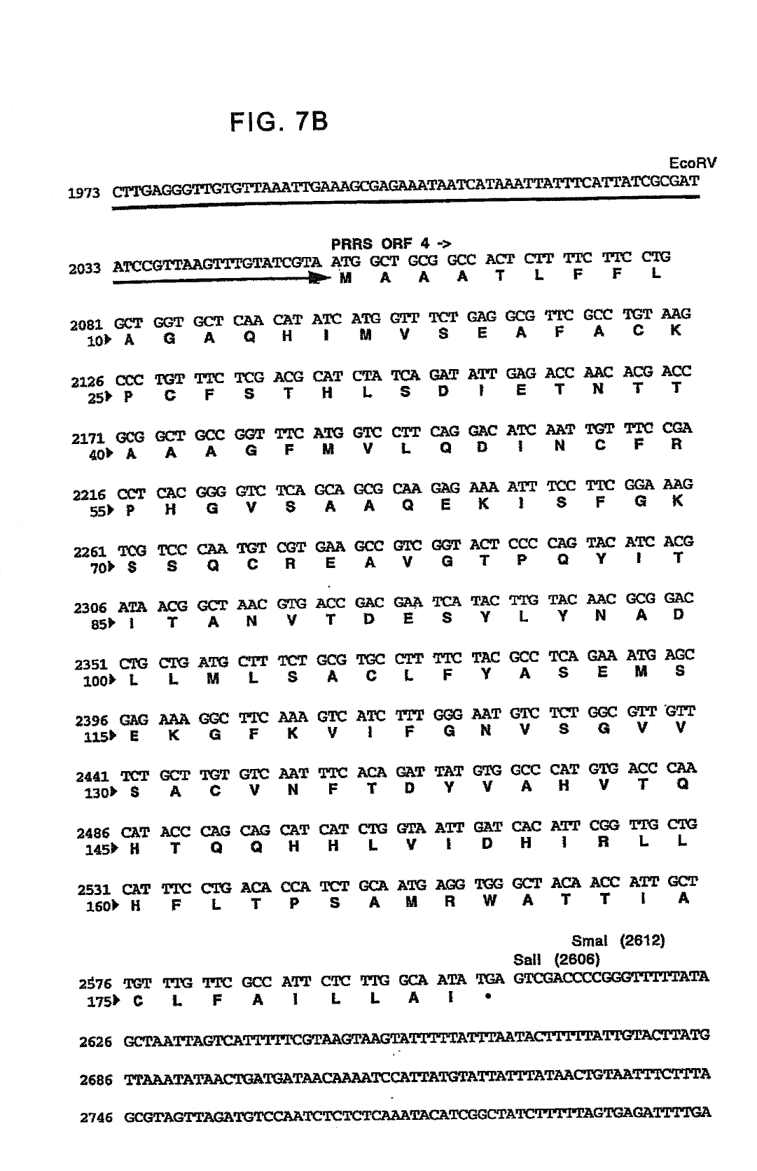

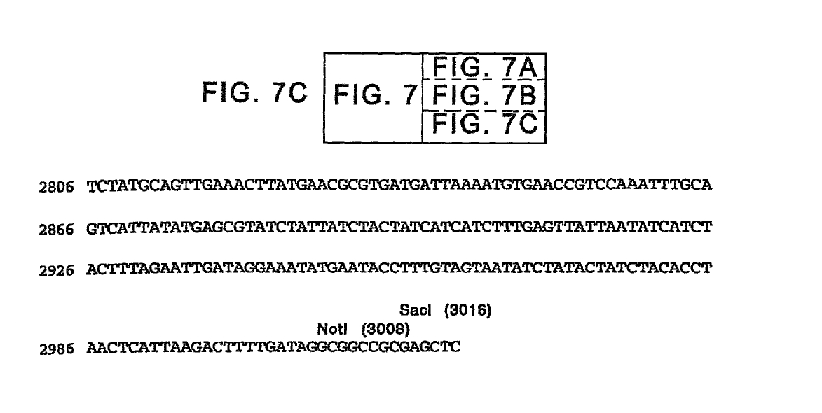

To insert PRRSV ORF 4 into the ALVAC C6 insertion plasmid, pJPO99 (see example 1), PCR J 1306 is generated using primers JP762 (SEQ ID NO: 17; contains the 3′ end of the H6 promoter (from the EcoRV site) and the 5′ end of PRRS ORF4) and JP763 (SEQ ID NO: 18; contains the 3′ end of PRRS ORF4 and a SalI cloning site) on plasmid pPB272. PCR J1306 is digested with EcoRV/SalI and the resulting ˜570bp fragment cloned into a ˜4.5 kb EcoRV/SalI band from pJP099. The resulting plasmid, designated pJP 103, is confirmed by sequence analysis to contain the same deduced amino acid sequence of ORF 4 as pPB272 (see the map in FIG. 6 and the sequence (SEQ ID NO: 19) in FIG. 7). This donor plasmid pJP103 (linearized with NotI) is used in an in vitro recombination (IVR) assay to generate ALVAC recombinant vCP1618 (see example 10).

Sequence of the Primers

Primer PB471 (SEQ ID NO:16)
TTG TCG ACG GCA ATT GGT TCC ATT TGG AAT G
Primer PB472 (SEQ ID NO:15)
TTG GAT CCC CAA TTT GTG AGA ACA TCT C
Primer JP762 (SEQ ID NO:17)
CAT CAT CAT GAT ATC CGT TAA GTT TGT ATC GTA ATG GCT GCG GCC ACT CTT
TTC
Primer JP763 (SEQ ID NO:18)
TAC TAC TAC GTC GAC TCA TAT TGC CAA GAG AAT GGC

### Example 6

Construction of Alvac Donor Plasmid for PRRSV ORF 5

First strand cDNA synthesis is performed in 20 μl final volume consisting of 1 μl of viral RNA (see example 2) and 19 μl of RT-PCR MasterMix according to 1^{st} Strand cDNA synthesis Kit (Perkin Elmer, manufactured by Roche Molecular Systems Inc., Branchburg, N.J., U.S.A.; Cat#N808-0017). The MasterMix includes MgCl₂ (5 mM), PCR bufferII (1×), dNTPs (1 mM), Rnase inhibitor (1 U), Murine Leukemia Virus Reverse Transcriptase (2.5 U), and oligonucleotide PB43 (0.75 μM) used as a primer (SEQ ID NO: 20). Reaction mixture is successively incubated at 42° C. for 15 min, 99° C. for 5 min and 4° C. for 5 min. The single strand cDNA is sub PCR-amplified in a 100 μl final volume consisting of the 20 μl RT-PCR reaction and 80 μl of PCR MasterMix (2 mM MgCl₂, PCR bufferII 1× and 2.5 U Ampli Taq® DNA Polymerase) including oligonucleotide primers PB462 (SEQ ID NO: 21) and PB43 (SEQ ID NO: 20). After a first 2 min. incubation at 95° C., 35 cycles of amplification (96° C. for 45 sec; 56° C. for 45 sec and 72° C. for 2 min) are performed. The PCR fragment containing the PRRSV ORF 5 coding sequence is purified by Geneclean (GENECLEAN Kit; BIO101 Vista, Calif., U.S.A.) and subsequently digested with SalI and BamHI to generate a SalI-BamHI fragment of 642bp. This fragment is cloned into the vector pVR1012 (VICAL Inc., San Diego, Calif., U.S.A.) digested with SalI and BamHI to generate plasmid pPB273. The PCR fragment is cloned into the vector pCRII (Invitrogen, Carlsbad, Calif., U.S.A.) to generated plasmid pPB267. Plasmid pPB273 is digested with SalI and ClaI to generate a SalI-ClaI fragment of 487bp (fragment A). Plasmid 267 is digested with ClaI and BamHI to generate a ClaI-BamHI fragment of 161bp (fragment B). Fragments A and B are ligated into vector pVRO12 (VICAL Inc., San Diego, Calif., U.S.A.) digested with SalI and BamHI to generate plasmid pPB270 which contains the PRRSV ORF 5. The ORF 5 present in three independent clones are sequenced in their entirety and a consensus sequence is established and found to be 100% homologous to the sequence of reference (PRRSV Lelystad strain, Genebank accession number M96262).

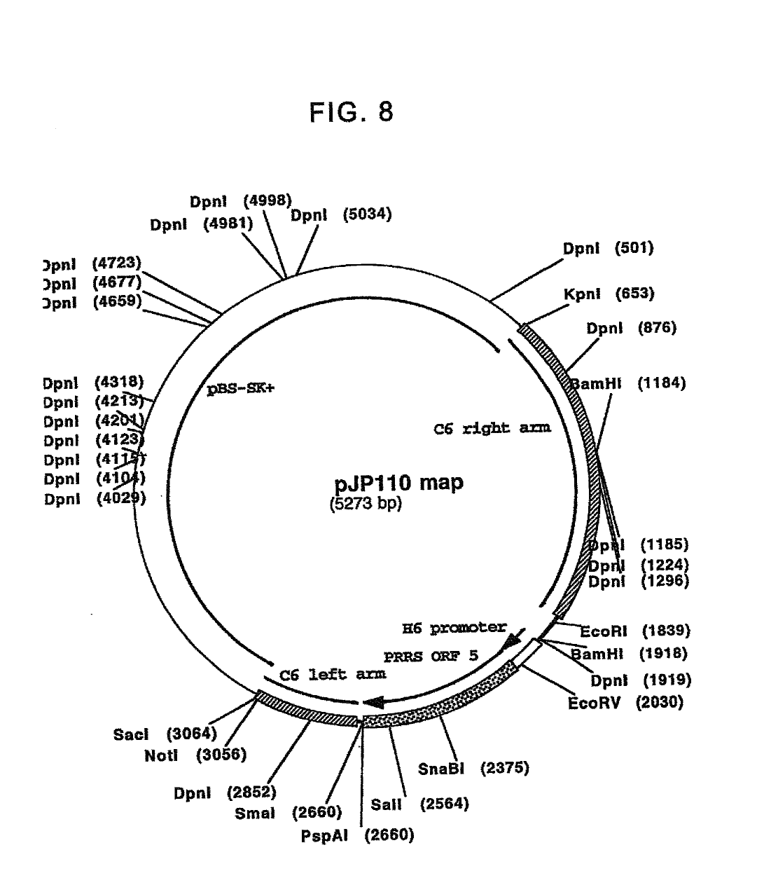

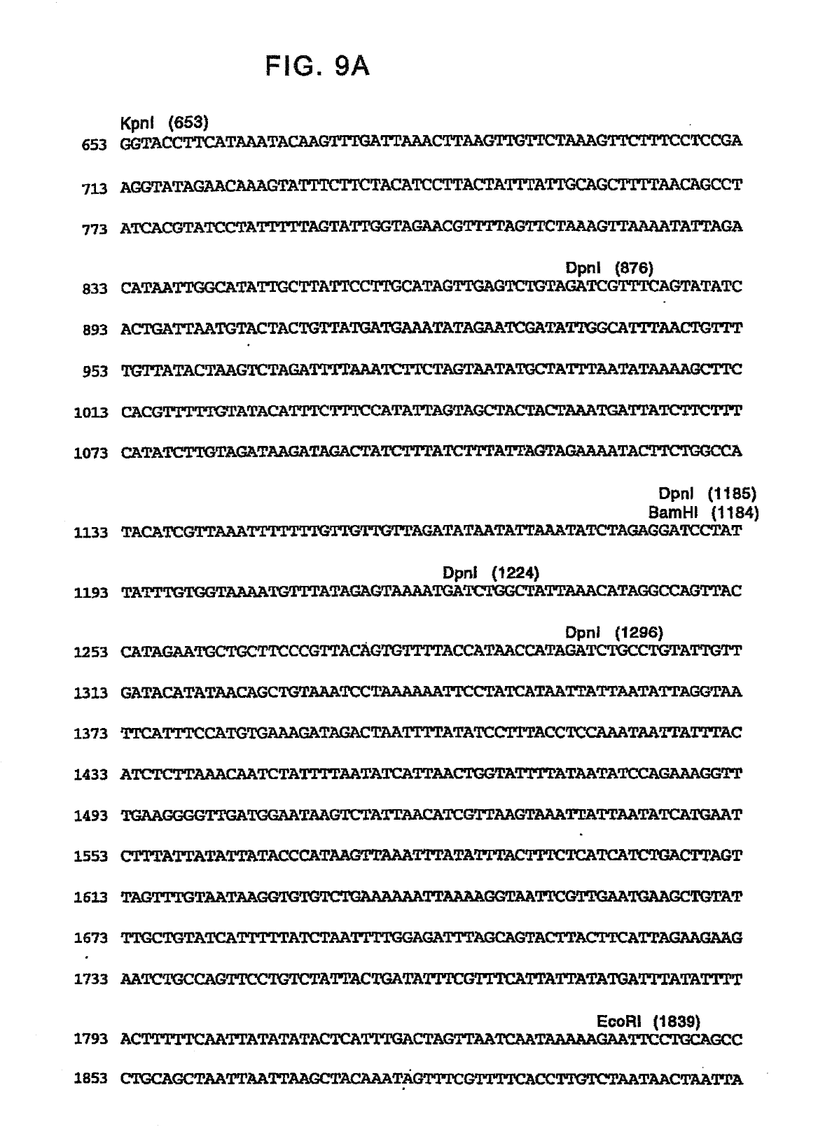

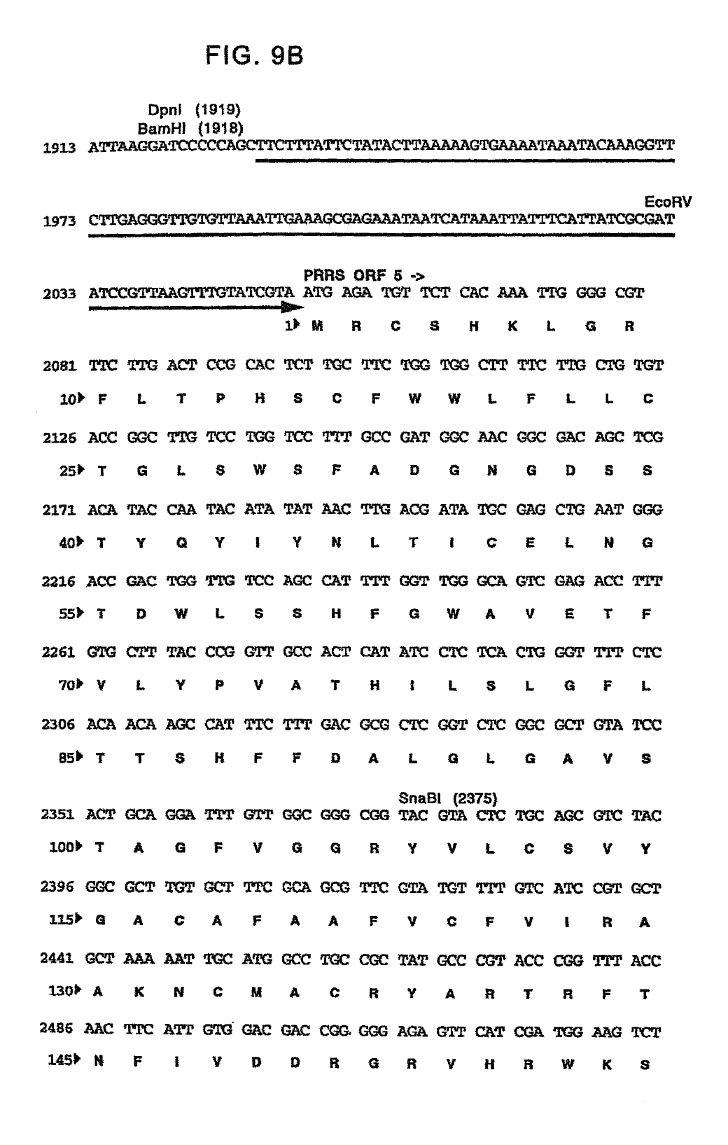

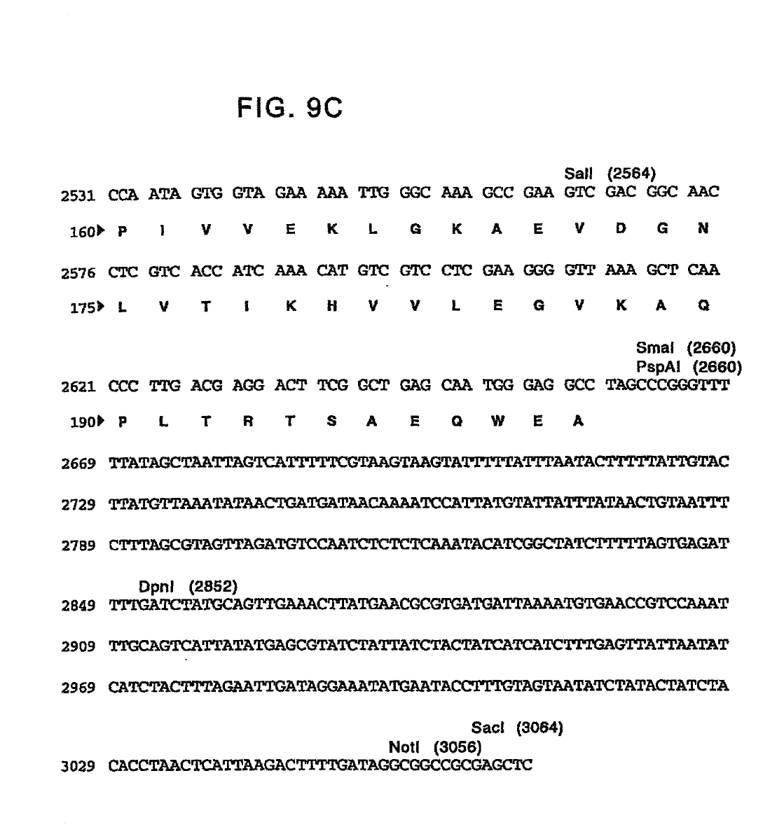

The sequence analysis of PRRS ORF 5 shows the presence of two T5NT at positions 59-65 and 264-270, which are known to be early transcription termination signals in poxvirus. In order to silently mutate these two T5NT encoded within ORF 5, the following strategy is employed. To mutate the T5NT (positions 59-65), primers JP764 (SEQ ID NO: 22; primer 764 contains the 3′ end of the H6 promoter and the first 71 bases of the 5′ end of the PRRS ORF 5 including the desired T5NT change) and JP776 (SEQ ID NO: 23; primer JP776 contains the 3′ end of PRRS ORF 5 and a PspAI cloning site) are used on plasmid pPB270 to generate ˜627bp PCR J1307, which is cloned into pCR2.1 plasmid (Invitrogen, Carlsbad, Calif.). The resulting plasmid is designated pJP106. A ˜627bp EcoRV/PspAI fragment, containing PRRS ORF 5, is isolated from plasmid pJP106, and cloned into a ˜4.5Kb EcoRV/PspAI fragment from pJP099 (see example 1) to create a new plasmid designated pJP108. To mutate the T5NT (positions 264-270), a ˜345bp fragment designated PCR J1314 is generated using primers JP766 (SEQ ID NO: 24; primer JP766 contains 20 bases of the H6 promoter) and JP767 (SEQ ID NO: 25; primer JP767 contains PRRS ORF 5 sequences including the desired T5NT change) on plasmid pJP108. PCR J 1314 is digested with EcoRV/SnaBI and cloned into a ˜4.9Kb EcoRV/SnaBI fragment from pJP108. The resulting donor plasmid, designated pJP110, is confirmed by sequence analysis to contain PRRS ORF 5 with the desired T5NT mutagenesis (T to C changes at positions 63 and 267 of the ORF), which do not effect the amino acid sequence of the gene (see the map in FIG. 8 and the sequence (SEQ ID NO: 26) in FIG. 9). This donor plasmid pJP110 (linearized with NotI) is used in an in vitro recombination (IVR) assay to generate ALVAC recombinant vCP1619 (see example 10).

Sequence of the Primers

Primer PB43 (SEQ ID NO:20)
ATA GGA TCC TTG CAA AAA TCG TCT AGG CC
Primer PB462 (SEQ ID NO:21)
TTG TCG ACG CCA TTC TCT TGG CAA TAT GAG ATG
Primer JP764 (SEQ ID NO:22)
ATC ATG ATA TCC GTT AAG TTT GTA TCG TAA TGA GAT GTT CTC ACA AAT TGG
GGC GTT TCT TGA CTC CGC ACT CTT GCT TCT GGT GGC TTT TCT TGC TGT G
Primer JP766 (SEQ ID NO:24)
ATT-TCA-TTA-TCG-CGA-TAT-CC
Primer JP767 (SEQ ID NO:25)
GCT-GCA-GAG-TAC-GTA-CCG-CCC-GCC-AAC-AAA-TCC-TGC-AGT-GGA-TAC-AGC-
GCC-GAG-ACC-GAG-CGC-GTC-AAA-GAA-ATG-GCT-TG
Primer JP776 (SEQ ID NO:23)
TAC-TAC-TAC-CCC-GGG-CTA-GGC-CTC-CCA-TTG-CTC-AGC

### Example 7

Construction of Alvac Donor Plasmid for PRRSV ORF 6

First strand cDNA synthesis is performed in 20 μl final volume consisting of 1 μl of viral RNA (see example 2) and 19 μl of RT-PCR MasterMix according to 1^{st} Strand cDNA synthesis Kit (Perkin Elmer, manufactured by Roche Molecular Systems Inc., Branchburg, N.J., U.S.A.; Cat#N808-0017). The MasterMix includes MgCl₂ (5 mM), PCR bufferII (1×), dNTPs (1 mM), Rnase inhibitor (1 U), Murine Leukemia Virus Reverse Transcriptase (2.5 U), and oligonucleotide PB465 (0.75 μM) used as a primer (SEQ ID NO: 27). Reaction mixture is successively incubated at 42° C. for 15 min, 99° C. for 5 min and 4° C. for 5 min. The single strand cDNA is subseq PCR-amplified in a 100 μl final volume consisting of the 20 μl RT-PCR reaction and 80 μl of PCR MasterMix (2 mM MgCl₂, PCR bufferII 1× and 2.5 U Ampli Taq® DNA Polymerase) including oligonucleotide primers PB464 (SEQ ID NO: 28) and PB465 (SEQ ID NO: 27). After a first 2 min. incubation at 95° C., 35 cycles of amplification (96° C. for 45 sec; 56° C. for 72° C. for 1.5 min) are performed. The PCR fragment containing the PRRSV ORF 6 coding sequence is purified by Geneclean (GENECLEAN Kit; BIO101, Vista, Calif., U.S.A.) and subsequently digested with SalI and BamHI to generate a SalI-BamHI fragment of 572bp. This fragment is cloned into the vector pVR1012 (VICAL Inc., San Diego, Calif., U.S.A.) digested with SalI and BamHI to generate plasmid pPB268. The ORF 6 present in one clone of pPB268 is found to be 100% homologous to the sequence of reference (PRRSV Lelystad strain, Genebank accession number M96262).

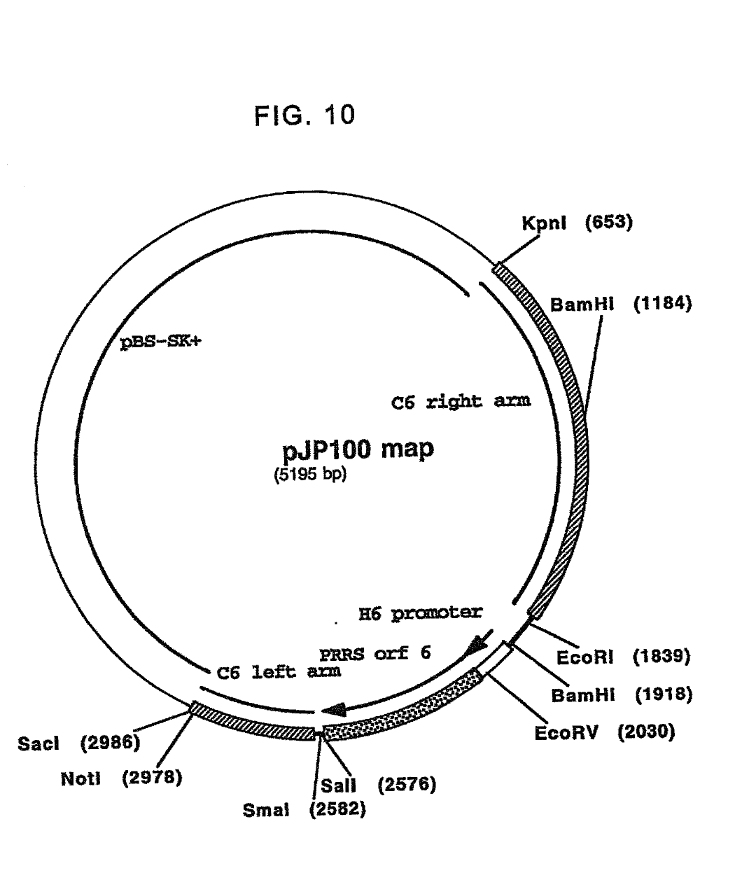

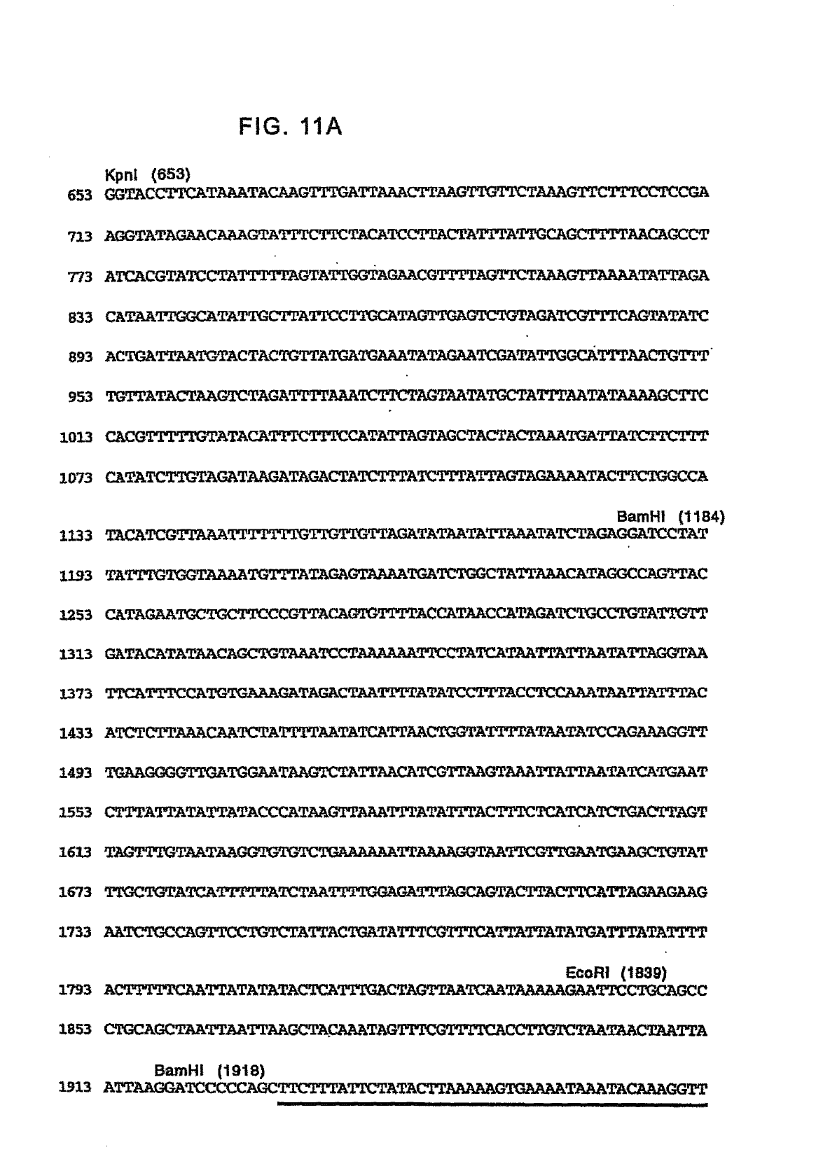

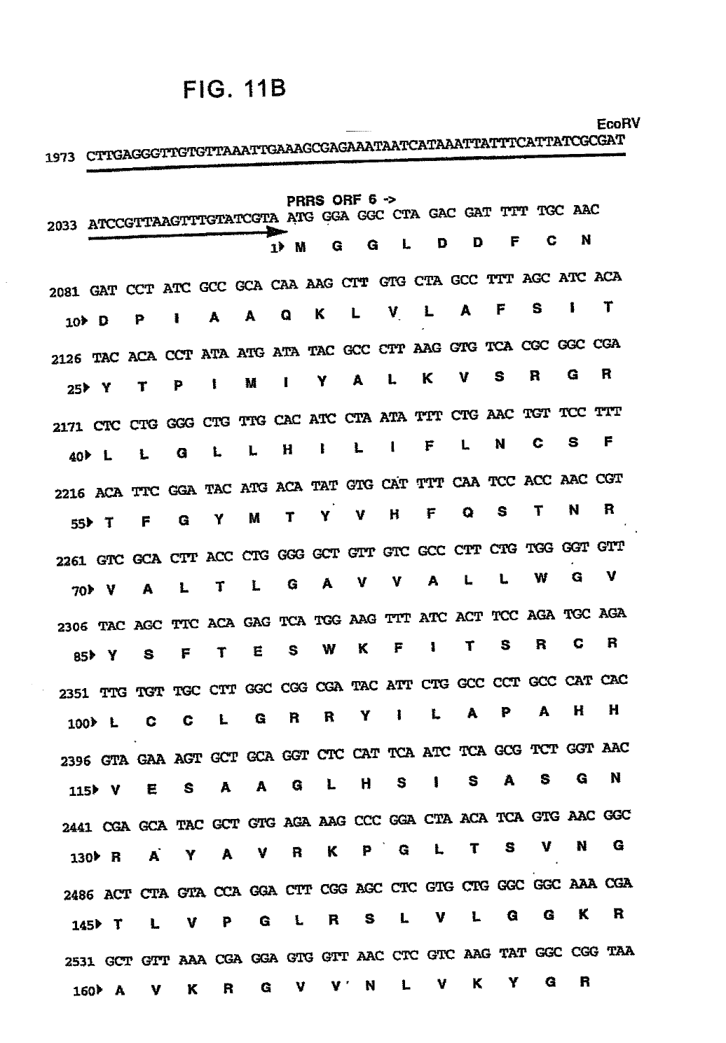

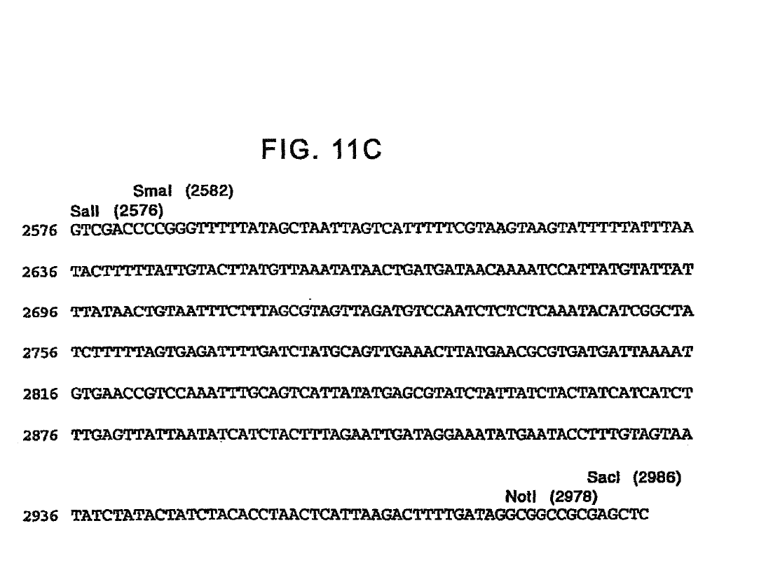

To insert PRRS ORF 6 into the ALVAC C6 insertion plasmid pJPO99 (see example 1), PCR J1302 is generated using primers JP768 (SEQ ID NO: 29; contains the 3′ end of the H6 promoter (from the EcoRV site) and the 5′ end of PRRS ORF 6) and JP769 (SEQ ID NO: 30; contains the 3′ end of PRRS ORF 6 and a SalI cloning site) on plasmid pPB268. PCR J1302 is digested with EcoRV/SalI and the resulting ˜550bp fragment cloned into a ˜4.5 kb EcoRV/SalI band from pJP099. The resulting plasmid is confirmed by sequence analysis and designated pJP100. Sequencing revealed 1 base change from the pPB268: a C to T change at position 51 that results in no amino acid change (see the map of pJP 100 in FIG. 10 and the sequence (SEQ ID NO: 31) in FIG. 11). This donor plasmid pJP100 (linearized with NotI) can be used in an in vitro recombination (IVR) assay to generate ALVAC recombinant using the method described in example 10.

Sequence of the Primers

Primer PB464 (SEQ ID NO:28)
TTG TCG ACG AGG ACT TCG GCT GAG CAA TG
Primer PB465 (SEQ ID NO:27)
TTG GAT CCT TTT CTT TTT CTT CTG GCT CTG G
Primer JP768 (SEQ ID NO:29)
CAT CAT CAT GAT ATC CGT TAA GTT TGT ATC GTA ATG GGA GGC CTA GAC GAT
TTT TG
Primer JP769 (SEQ ID NO:30)
TAC TAC TAC GTC GAC TTA CCG GCC ATA CTT GAC GAG

### Example 8

Construction of Alvac Double Donor Plasmid for PRRSV ORF 5 and ORF 6

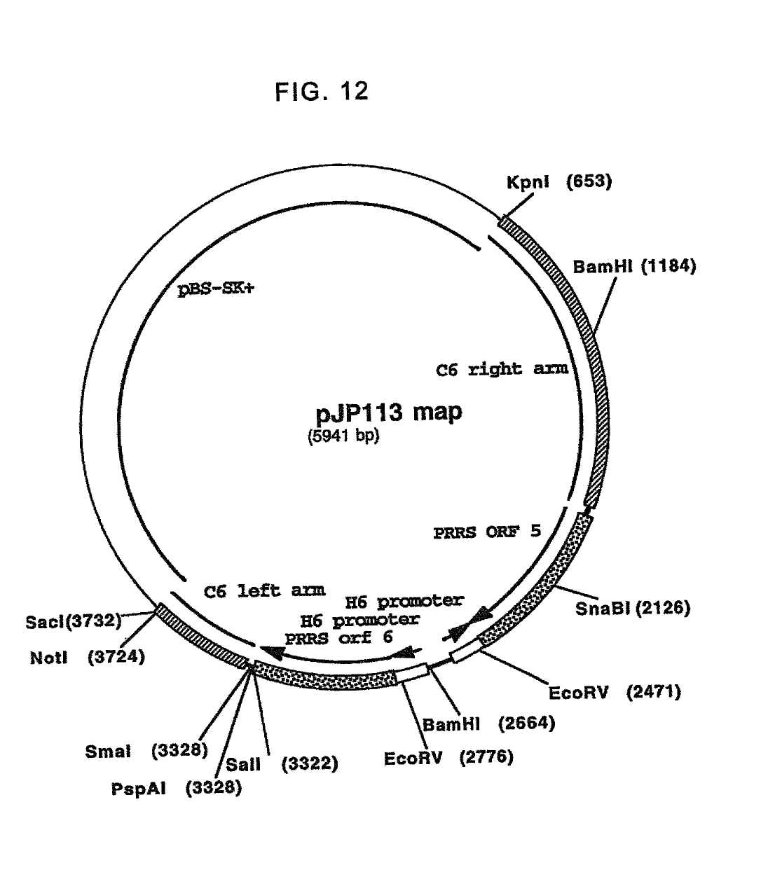

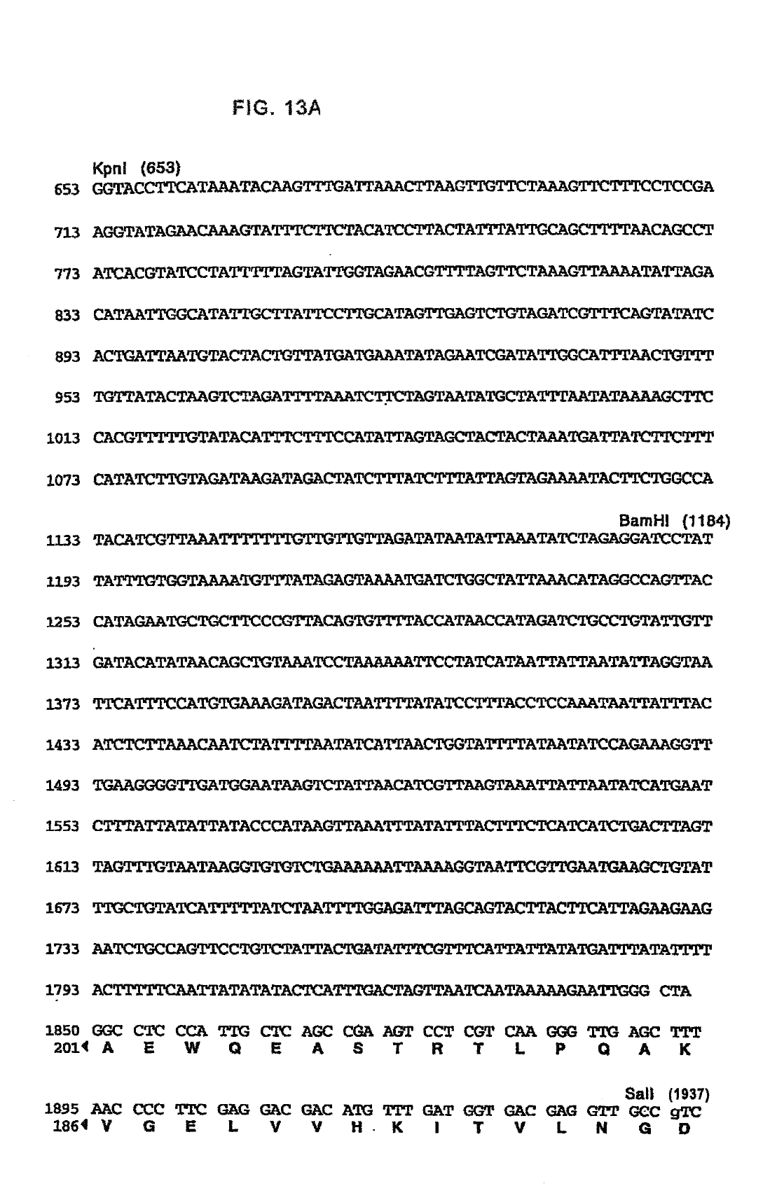

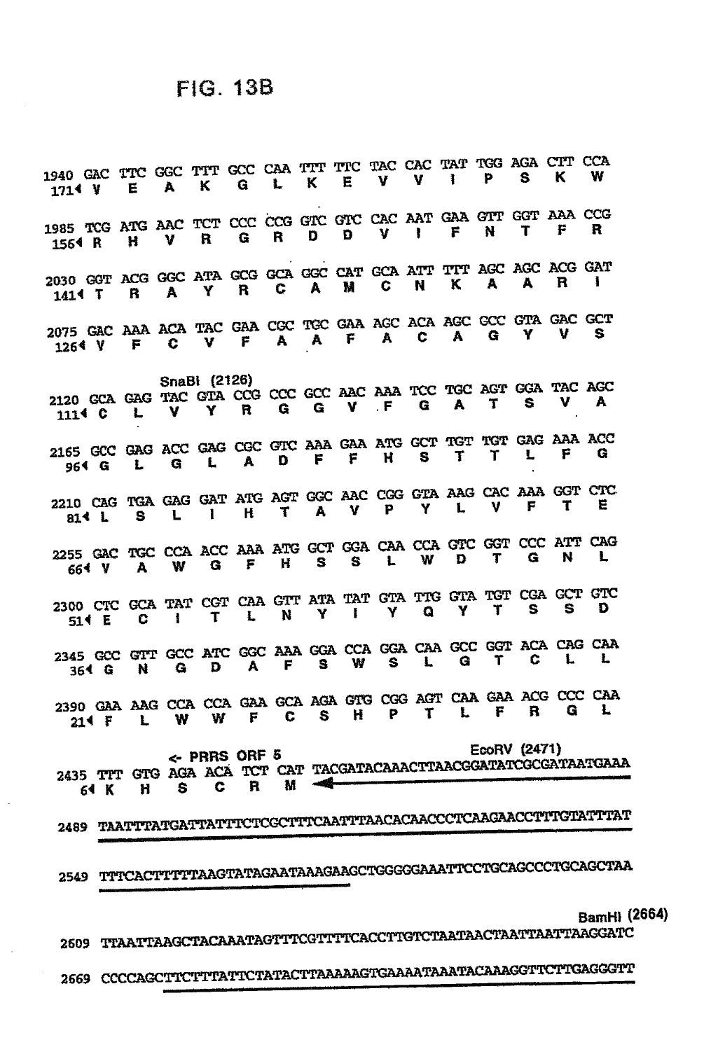

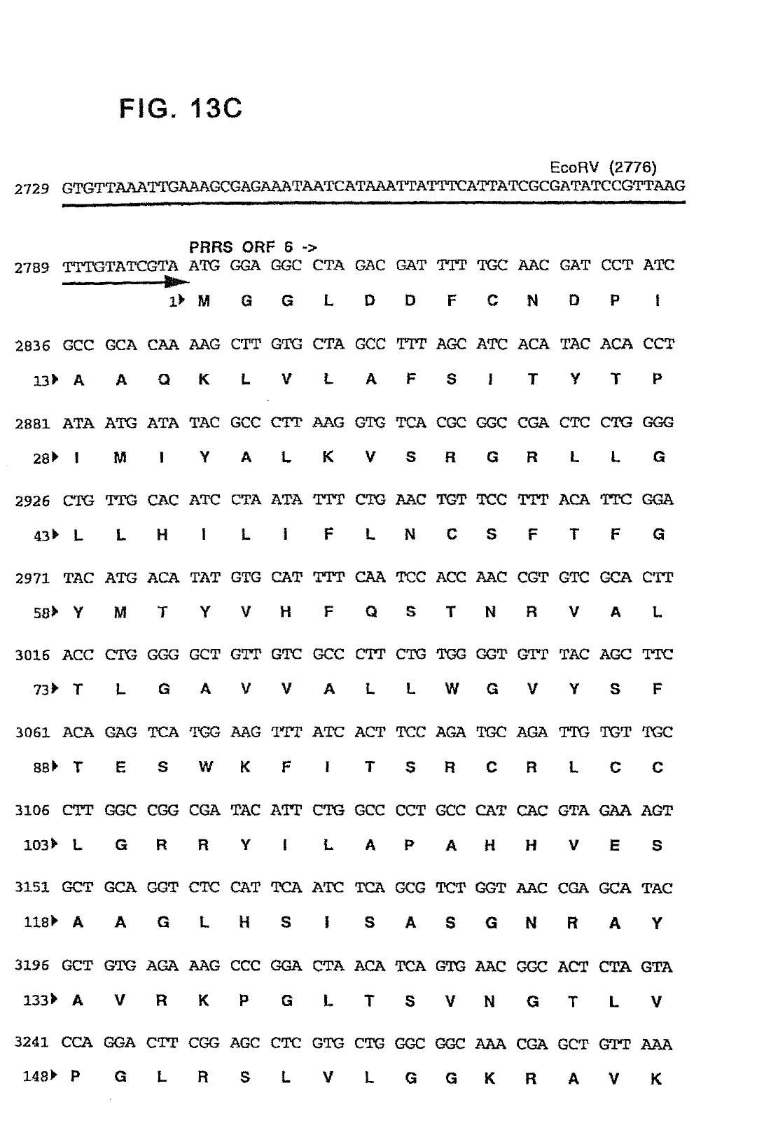

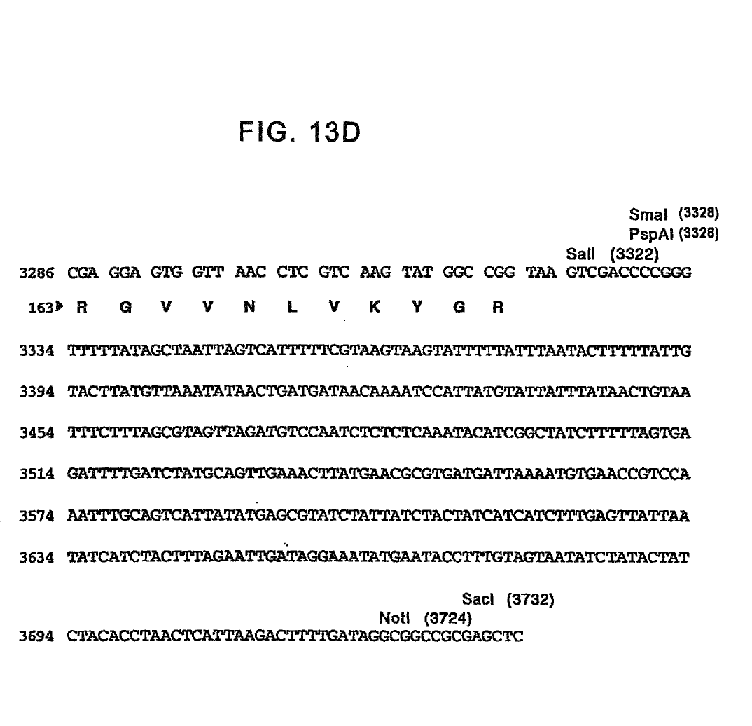

To insert ORF 5 into the ORF 6 donor plasmid in a head-to-head orientation (the 5′ end of the 2 promoters being linked, and the two ORFs being in opposite orientation; see FIG. 12), a ˜742bp SmaI/DpnI fragment from pJP 110 (see example 6) is cloned into SmaI digested pJP100 (see example 7). The resulting plasmid is confirmed by restriction analysis to contain the PRRS ORF 5 and 6 cassettes in the desired orientation and is designated pJP 113 (see the map in FIG. 12 and the sequence (SEQ ID NO: 32) in FIG. 13). This donor plasmid pJP113 (linearized with NotI) is used in an in vitro recombination (IVR) assay to generate ALVAC recombinant vCP1626 (see example 10).

### Example 9

Construction of Alvac Donor Plasmid for PRRSV ORF 7

First strand cDNA synthesis is performed in 20 μl final volume consisting of 1 μl of viral RNA (see example 2) and 19 μl of RT-PCR MasterMix according to ₁ st Strand cDNA synthesis Kit (Perkin Elmer, manufactured by Roche Molecular Systems Inc., Branchburg, N.J., U.S.A.; Cat#N808-0017). The MasterMix includes MgCl₂ (5 mM), PCR bufferII (1×), dNTPs (1 mM), Rnase inhibitor (1 U), Murine Leukemia Virus Reverse Transcriptase (2.5 U), and oligonucleotide PB461 (0.75 μM) used as a primer (SEQ ID NO: 33). Reaction mixture is successively incubated at 42° C. for 15 min, 99° C. for 5 min and 4° C. for 5 min. The single strand cDNA is subseq PCR-amplified in a 100 μl final volume consisting of the 20 μl RT-PCR reaction and 80 μl of PCR MasterMix (2 mM MgCl₂, PCR bufferII 1× and 2.5 U Ampli Taq® DNA Polymerase) including oligonucleotide primers PB460 (SEQ ID NO: 34) and PB461 (SEQ ID NO: 33). After a first 2 min incubation at 95° C., 35 cycles of amplification (96° C. for 45 sec; 56° C. for 72° C. for 1.5 min) are performed. The PCR fragment containing the PRRSV ORF 7 coding sequence is purified by Geneclean (GENECLEAN Kit; BIO101, Vista, Calif., U.S.A.) and subsequently digested with SalI and BamHI to generate a SalI-BamHI fragment of 411bp. This fragment is cloned into the vector pVR1 12 (VICAL Inc., San Diego, Calif., U.S.A.) digested with SalI and BamHI to generate plasmid pPB269. The ORF 6 present in one clone of pPB269 is found to be 100% homologous to the sequence of reference (PRRSV Lelystad strain, Genebank accession number M96262).

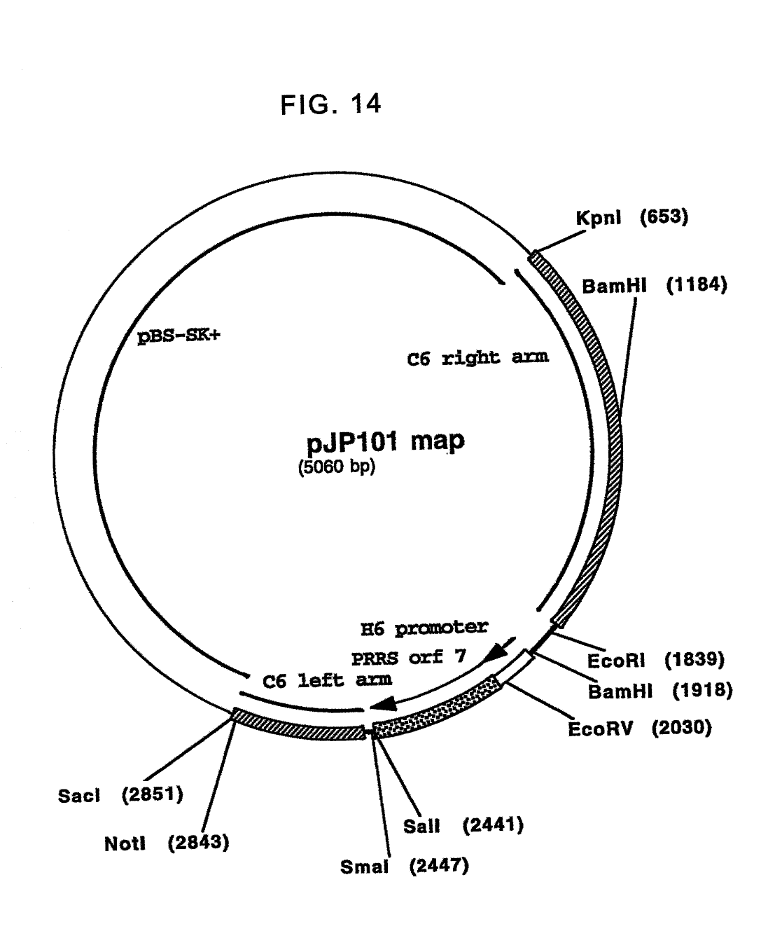

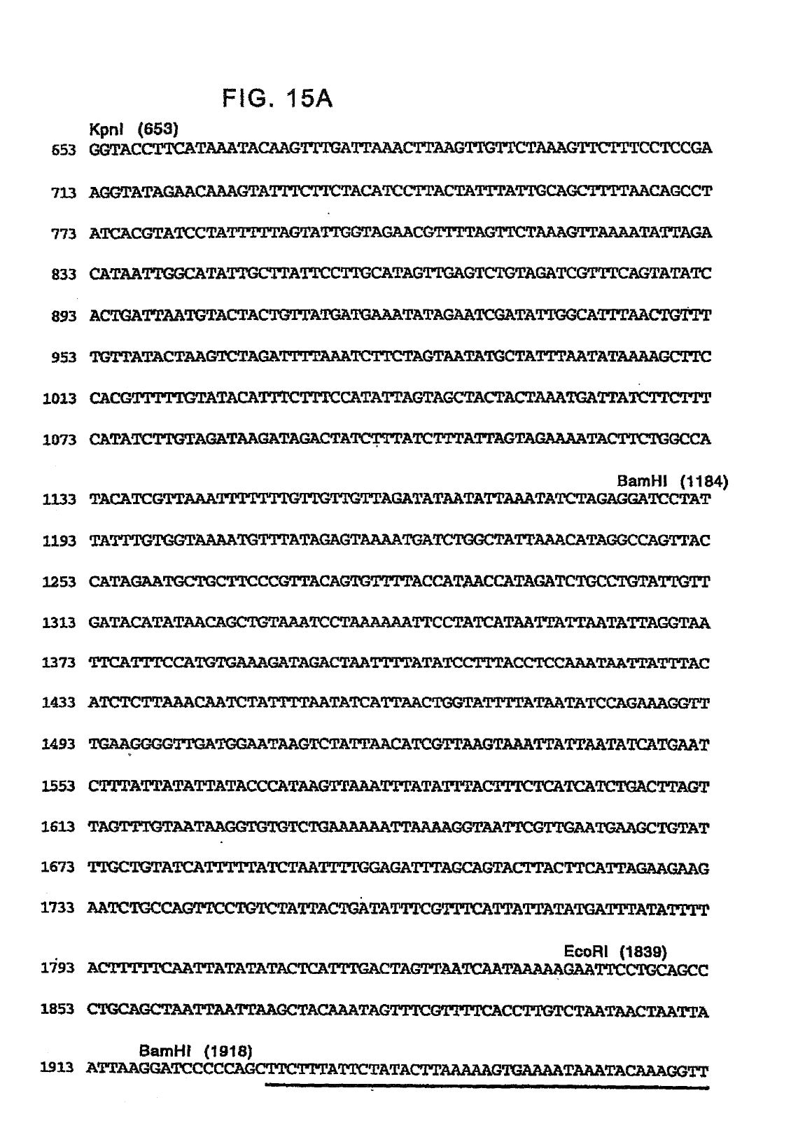

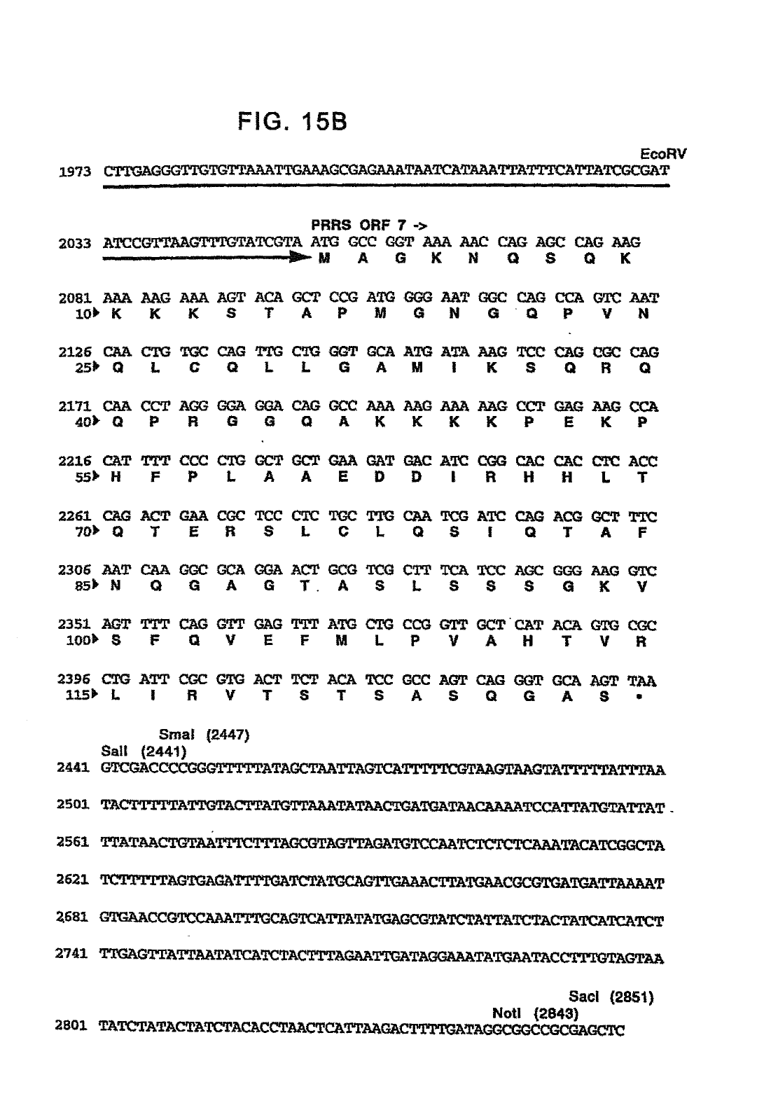

To insert PRRS ORF 7 into the ALVAC C6 insertion plasmid pJPO99 (see example 1), PCR J1303 is generated using primers JP770 (SEQ ID NO: 35; contains the 3′ end of the H6 promoter (from the EcoRV site) and the 5′ end of PRRS ORF 7) and JP771 (SEQ ID NO: 36; contains the 3′ end of PRRS ORF 7 and a SalI cloning site) on plasmid pPB269. PCR J1303 is digested with EcoRV/SalI and the resulting ˜415bp fragment cloned into a ˜4.5 kb EcoRV/SalI band from pJPO99. The resulting plasmid is confirmed by sequence analysis and designated pJP101 (see the map in FIG. 14 and the sequence (SEQ ID NO: 37) in FIG. 15). This donor plasmid pJP 101 (linearized with NotI) can be used in an in vitro recombination (IVR) assay to generate ALVAC recombinant using the method described in example 10.

Sequence of the Primers

Primer PB460 (SEQ ID NO:34)
TTG TCG ACA TGG CCG GTA AAA ACC AGA GCC
Primer PB461 (SEQ ID NO:33)
TTG GAT CCA TTC ACC TGA CTG TCA AAT TAA C
Primer JP770 (SEQ ID NO:35)
CAT CAT CAT GAT ATC CGT TAA GTT TGT ATC GTA ATG GCC GGT AAA AAC CAG
AGC
Primer JP771 (SEQ ID NO:36)
TAC TAC TAC GTC GAC TTA ACT TGC ACC CTG ACT GGC

### Example 10

Generation of Alvac-PRRSV Recombinants

Plasmids pJP115 (ORF 2; see example 3), pJP 119 (ORF 3; see example 4), pJP103 (ORF 4; see example 5), pJP110 (ORF 5; see example 6), and pJP1 13 (ORF 5 and ORF 6; see example 8) are linearized with NotI and transfected into ALVAC infected primary CEF cells by using the calcium phosphate precipitation method previously described (Panicali and Paoletti, 1982; Piccini et al., 1987). Positive plaques are selected on the basis of hybridization to specific PRRSV radiolabeled probes and subjected to four sequential rounds of plaque purification until a pure population is achieved. One representative plaque from each IVR is then amplified and the resulting ALVAC recombinants are designated vCP1642 (ORF2), vCP1643, (ORF 3), vCP1618 (ORF 4), vCP 1619 (ORF 5), and vCP 1626 (ORF 5 and ORF 6). Table 1 indicates the name of the donor plasmids, ALVAC recombinants and expressed PRRS ORFs. All these recombinants are the result of recombination events between the ALVAC vector and the donor plasmids, and they contain PRRSV ORF(s) inserted into the ALVAC C6 locus. The genomic structure is determined for all recombinants by restriction analysis and Southern blotting using different probes.

TABLE 1
List of generated ALVAC-PRRSV recombinants

| Name of | Name of | Expressed |
|---|---|---|
| donor plasmid | ALVAC recombinant | PRRSV ORF(s) |
| pJP115 | VCP1642 | ORF 2 |
| pJP119 | VCP1643 | ORF 3 |
| pJP103 | VCP1618 | ORF 4 |
| pJP110 | VCP1619 | ORF 5 |
| pJP113 | VCP1626 | ORF 5 and ORF6 |

In a similar fashion, recombinant ALVAC expressing only PRRSV ORF 6 and PRRSV ORF 7 can be generated using the donor plasmids pJP100 and pJP101, described in example 7 and 9, respectively.

### Example 11

Formulation of Recombinant Canarypox Viruses with Carbopol™ 974P

For the preparation of vaccines, recombinant canarypox viruses (example 10) can be mixed with solutions of carbomer. The carbomer component used for vaccination of pigs according to the present invention is the Carbopol™ 974P manufactured by the company BF Goodrich (molecular weight of 3,000,000). A 1.5 % Carbopol™ 974P stock solution is first prepared in distilled water containing 1 g/l of sodium chloride. This stock solution is then used for manufacturing a 4 mg/ml Carbopol™ 974P solution in physiological water. The stock solution is mixed with the required volume of physiological water, either in one step or in several successive steps, adjusting the pH value at each step with a IN (or more concentrated) sodium hydroxide solution to get a final pH value of 7.3-7.4. This final Carbopol™ 974P solution is a ready-to-use solution for reconstituting a lyophilized recombinant virus or for diluting a concentrated recombinant virus stock. For example, to get a final viral suspension containing 10^{e}8 pfu per dose of 2 ml, one can dilute 0.1 ml of a 10^{e}9 pfu/ml viral stock solution into 1.9 ml of the above Carbopol™ 974P 4 mg/ml ready-to-use solution. The same calculation applies in the case of mixtures of recombinant viruses, by taking into account the final pfu/ml titers that need to be achieved. In the same fashion, Carbopol™ 974P 2 mg/ml ready-to-use solutions can also be prepared.

### Example 12

Vaccination/Challenge in the Pregnant sow Model

Conventional gilts of 8 months of age are vaccinated with individual ALVAC/PRRSV recombinants or with mixtures of ALVAC/PRRSV recombinants. Vaccine viral suspensions are prepared by dilution of recombinant viruses stocks in sterile physiological water (NaCl 0.9 %). Suitable ranges for viral suspensions can be approximately 10^{e}6, 10^{e}7, 10^{e}8 pfu/dose. Vaccine solutions can also be prepared by mixing the recombinant virus suspension with a solution of Carbopol™ 974P as described in Example 11.

Individual recombinant viruses or mixture of recombinant viruses can be incorporated in the vaccines. For instance, vaccines may be composed of vCP1642,vCP1643,vCP1618, vCP1619, vCP1626 diluted in physiological water, or of mixtures of vCP1643+vCP1619, vCP1643+vCP1626, vCP1618+vCP 1626, vCP 1619+vCP 1618 diluted in physiological water. As described above (example 11), the same individual recombinant viruses or mixtures of recombinant viruses can be mixed with a Carbopol™ 974P solution.

All vaccines are injected intramuscularly under a volume of 2 ml.

The first vaccination is given at day 0, and a boost is given approximately 21 days after the first injection.

One group of control sows is not vaccinated (group not vaccinated and challenged).

The gilts are then vaccinated with standard vaccines, treated with the appropriate hormone regimen, observed for estral signs, and inseminated around day 37 with semen obtained from PRRSV-free boars. The pregnant sows (confirmation by ultrasound) are then free-housed in large pens with straw for bedding. They are fed with standard feed and have access ad libitum to water.

Around day 127 (approximately 90 days of gestation), all groups of sows are challenged by an intranasal administration of 1 ml of a suspension of the PRRSV P120 117B challenge strain (viral titer of the suspension=approximately 10^{e}7,5 CCID₅₀). The challenge virus is administered by spray in each nostril, using a syringe.

All animals are monitored post-challenge on the following criteria:

rectal temperature from the day before and after challenge;

weight of piglets at birth, around day 7, day 14 and day 21 of age;

abnormal behavior.

Blood samples are collected from sows throughout the experiment for viral isolation and antibody titration.

Samples of colostrum (at farrowing) and milk are also collected.

Blood samples are collected from piglets throughout the experiment for viral isolation and antibody titration

Post-mortem examinations are carried out for each animal that dies or is euthanized.

Inventive recombinants elicit an immunological response.

### Example 13

Vaccination/Challenge in the Piglet Model

Conventional piglets of 8-10 weeks of age and of 25-27 kg in average, obtained from a free-PRRSV status breeding farm, are weighed and randomly grouped according to their weight. Vaccinated groups are constituted by 6 piglets and one control group (non vaccinated) is constituted with 9 piglets. They are vaccinated with individual ALVAC/PRRSV recombinants or with mixtures of ALVAC/PRRSV recombinants. Vaccine viral suspensions are prepared by dilution of recombinant viruses stocks in sterile physiological water (NaCl 0.9 %). Suitable ranges for viral suspensions can be approximately 10^{e}6, 10^{e}7, 10^{e}8 pfu/dose. Vaccine solutions can also be prepared by mixing the recombinant virus suspension with a solution of Carbopol™ 974P as described in Example 11.

Individual recombinant viruses or mixture of recombinant viruses can be incorporated in the vaccines. For instance, vaccines may be composed of vCP1642,vCP1643,vCP1618, vCP1619, vCP1626 diluted in physiological water, or of mixtures of vCP1643+vCP1619, vCP1643+vCP 1626, vCP 1618+vCP 1626, vCP 1619+vCP 1618 diluted in physiological water. As described above (example 11), the same individual recombinant viruses or mixtures of recombinant viruses can be mixed with a Carbopol™ 974P solution.

The piglets are vaccinated at day 0 with a single dose of 2 ml of vaccine by the intramuscular route, and they are boosted with a single dose of 2 ml of the same vaccine by the intramuscular route approximately 21 days after the first injection.

All groups of piglets, including the non vaccinated group, are challenged around day 35, by an intranasal administration of approximately 1.5 ml of a suspension of the PRRSV challenge strain (P120 117B strain) with a titer of approximately 10^{e}6,5 CCID₅₀ per ml. The challenge virus is administered by spray in each nostril, using a syringe.

After challenge, all piglets are monitored for clinical signs (diarrhea, anorexia, depression/prostration, vomit, cough/sneeze, conjunctivitis), for rectal temperature and for weight.

All piglets are weighed around day -1, day 21, day 35 (day of challenge) and day 56 (end of experiment; euthanasia of piglets).

Blood samples are taken for viral isolation and antibody titration.

After euthanasia, all piglets are necropsied and samples of lungs are taken for viral isolation.

Inventive recombinants elicit an immunological response.

The invention will be further described in the following numbered paragraphs:

1. A recombinant avipox virus comprising DNA complementary to genomic RNA from porcine reproductive and respiratory syndrome virus (PRRSV), or a recombinant vector comprising DNA complementary to genomic RNA from PRRSV ORFs 3 and 5, or 5 and 6, or 4 and 5, or 3 and 5 and 6, or 4 and 5 and 6.

2. The recombinant avipox virus of paragraph 1 which is ALVAC.

3. The recombinant ALVAC virus of paragraph 2, wherein the DNA complementary to genomic RNA from porcine reproductive and respiratory syndrome virus codes for and is expressed as PRRSV glycoproteins, membrane or capsid proteins.

4. The recombinant ALVAC virus of paragraph 3, wherein the DNA complementary to genomic RNA from porcine reproductive and respiratory syndrome virus corresponds to the open reading frame 2 (ORF 2) of porcine reproductive and respiratory syndrome virus.

5. The recombinant ALVAC virus of paragraph 3, wherein the DNA complementary to genomic RNA from porcine reproductive and respiratory syndrome virus corresponds to the open reading frame 3 (ORF 3) of porcine reproductive and respiratory syndrome virus.

6. The recombinant ALVAC virus of paragraph 3, wherein the DNA complementary to genomic RNA from porcine reproductive and respiratory syndrome virus corresponds to the open reading frame 4 (ORF 4) of porcine reproductive and respiratory syndrome virus.

7. The recombinant ALVAC virus of paragraph 3, wherein the DNA complementary to genomic RNA from porcine reproductive and respiratory syndrome virus corresponds to the open reading frame 5 (ORF 5) of porcine reproductive and respiratory syndrome virus.

8. The recombinant ALVAC virus of paragraph 3, wherein the DNA complementary to genomic RNA from porcine reproductive and respiratory syndrome virus corresponds to the open reading frame 6 (ORF 6) of porcine reproductive and respiratory syndrome virus.

9. The recombinant ALVAC virus of paragraph 3, wherein the DNA complementary to genomic RNA from porcine reproductive and respiratory syndrome virus corresponds to the open reading frame 7 (ORF 7) of porcine reproductive and respiratory syndrome virus.

10. The recombinant ALVAC virus of paragraph 3, wherein the DNA complementary to genomic RNA from porcine reproductive and respiratory syndrome virus corresponds to the open reading frames 5 and 6 (ORF 5 and 6) of porcine reproductive and respiratory syndrome virus.

11. The recombinant ALVAC virus of paragraph 10, wherein ORF 5 and ORF 6 are in a head-to-head orientation.

12. The recombinant vector of paragraph 1 wherein the vector comprises an ALVAC virus that contains and expresses PRRSV ORFs 3 and 5, or 5 and 6, or 4 and 5,or 3 and 5 and 6, or 4 and 5 and 6.

13. The recombinant ALVAC virus of paragraph 3 which is vCP1618, vCP1619, vCP1626, or vCP1643.

14. An immunological composition for inducing an immunological response in a host inoculated with the immunological composition, the immunological composition comprising a carrier and the recombinant virus of paragraph 1.

15. An immunological composition for inducing an immunological response in a host inoculated with the immunological composition, the immunological composition comprising a carrier and the recombinant virus of paragraph 14.

16. An immunological composition for inducing an immunological response in a host inoculated with the immunological composition, the immunological composition comprising a carrier and the recombinant virus of paragraph 4.

17. An immunological composition for inducing an immunological response in a host inoculated with the immunological composition, the immunological composition comprising a carrier and the recombinant virus of paragraph 5.

18. An immunological composition for inducing an immunological response in a host inoculated with the immunological composition, the immunological composition comprising a carrier and the recombinant virus of paragraph 6.

19. An immunological composition for inducing an immunological response in a host inoculated with the immunological composition, the immunological composition comprising a carrier and the recombinant virus of paragraph 7.

20. An immunological composition for inducing an immunological response in a host inoculated with the immunological composition, the immunological composition comprising a carrier and the recombinant virus of paragraph 8.

21. An immunological composition for inducing an immunological response in a host inoculated with the immunological composition, the immunological composition comprising a carrier and the recombinant virus of paragraph 9.

22. An immunological composition for inducing an immunological response in a host inoculated with the immunological composition, the immunological composition comprising a carrier and the recombinant virus of paragraph 10.

23. An immunological composition for inducing an immunological response in a host inoculated with the immunological composition, the immunological composition comprising a carrier and the recombinant virus of paragraph 11.

24. An immunological composition for inducing an immunological response in a host inoculated with the immunological composition, the immunological composition comprising a carrier and the recombinant virus of paragraph 12.

25. A method for inducing an immunological response in a host comprising administering to the host the immunological composition of paragraph 15.

26. A method for inducing an immunological response in a host comprising administering to the host the immunological composition of paragraph 16.

27. A method for inducing an immunological response in a host comprising administering to the host the immunological composition of paragraph 17.

28. A method for inducing an immunological response in a host comprising administering to the host the immunological composition of paragraph 18.

29. A method for inducing an immunological response in a host comprising administering to the host the immunological composition of paragraph 19.

30. A method for inducing an immunological response in a host comprising administering to the host the immunological composition of paragraph 20.

31. A method for inducing an immunological response in a host comprising administering to the host the immunological composition of paragraph 21.

32. A method for inducing an immunological response in a host comprising administering to the host the immunological composition of paragraph 22.

33. A method for inducing an immunological response in a host comprising administering to the host the immunological composition of paragraph 23.

34. A method for inducing an immunological response in a host comprising administering to the host the immunological composition of paragraph 24.

35. A method for inducing an immunological response in a host comprising administering to the host the immunological composition of paragraph 25.

Having thus described in detail preferred embodiments of the present invention, it is to be understood that the invention defined by the appended paragraphs is not to be limited to particular details set forth in the above description as many apparent variations thereof are possible without departing from the spirit or scope of the present invention.

### REFERENCES

1. Albina, E. 1997. Epidemiology of porcine reproductive and respiratory syndrome (PRRS): An overview. Veterinary Microbiology 55:309-316.

2. Bautista, E. M., Morrison, R. B., Goyal, S. M., Collins, J. E. and Annelli, J. F. 1993. Seroprevalence of PRRS virus in the United States. Swine Health Prod. 1(6): 4-7.

3. Bautista, E. M., Suarez P., Molitor T. W. 1999. T cell response to the structural polypeptides of porcine reproductive and respiratory syndrome virus. Arch. Virol. 144:117-134.

4. Benfield, D. A., Nelson, E. A., Collins, J. E., Harris, L., Goyal, S. M., Robinson, D., Christianson, W. T., Morrison, R. B., Gorcyca, D. and Chladek, D. 1992. Characterization of swine infertility and respiratory syndrome (SIRS) virus (isolate ATCC VR-2332). J.

Vet. Diagn. Invest. 4:127-133.

5. Brierley, I. 1995. Ribosomal frameshifting on viral RNAs. J. Gen. Virol. 76:1885-1892.

6. Brun A., Vaganay, A., Tardy, M. C., Noe, T., Vandeputte, J., Schirvel, C. and Lacoste, F. 1992. Evaluation of etiological elements in the “P.R.R.S.” in pigs. In Proceedings of the 12^{th} Congress of the International Pig Veterinary Society, The Hague, Netherlands, 17-20 August, p. 108.

7. Carlson, J. 1992. Encephalomyocarditis virus (EMCV) as a cause of reproductive and respiratory disease in swine. American Association of Swine Practitioners Newsletter. 4:23.

8. Cavanagh, D. 1997. Nidovirales: a new order comprising Coronaviridae and Arteriviridae. Arch Virol. 142 (3): 629-633.

9. Cho, S. H., Freese, W. R., Yoon, I. J., Trigo, A. V. and Joo, H. S. 1993. Seroprevalence of indirect fluorescent antibody to porcine reproductive and respiratory syndrome virus in selected swine herds. J. Vet. Diagn. Invest. 5:259-260.

10. Collins, J. E., Benfield, D. A., Christianson, W. T., Harris, L., Hennings, J. C., Shaw, D. P., Goyal, S. M., McCulloygh, S., Morrisson, R. B., Joo, H. S., Gorcyca, D. and Chladek, D. W. 1992. Isolation of swine infertility and respiratory syndrome virus (isolate ATCC VR-2332) in North America and experimental reproduction of the disease in gnotobiotic pigs. J. Vet. Diagn. Invest. 4:117-126.

11. Conzelmann, K. K., Visser, N., van Woensel, P. and Tiel, H. J. 1993. Molecular characterization of porcine reproductive and respiratory syndrome virus, a member of the Arterivirus group. Virology 193:329-339.

12. Den Boon, J. A., Snijder, E. J., Chirnside, E. D., de Vries, A. A. F., Horzinek, M. C. and Spaan, W. 1991. Equine arteritis virus is not a togavirus but belongs to the coronavirus superfamily. J. Virol. 65:2910-2920.

13. De Vries, A. A. F., Horzinek, M. C., Rottier, P. J. M. and de Groot, R. J. 1997. The genome organization of the Nidovirales: Similiarities and differences between Arteri-, Toro-, and Coronaviruses. Seminars in Virology 8:33-47.

14. Dewey CE, Wilson S, Buck P, Leyenaar JK. 1999. The reproductive performance of sows after PRRS vaccination depends on stage of gestation. Prev Vet Med 40:233-241.

15. Done, S. H. and Paton, D. J. 1995. Porcine reproductive and respiratory syndrome: clinical disease, pathology and immunosuppression. Veterinary Record 136:32-35.

16. Done, S. H., Paton, D. J. and White, M. E. C. 1996. Porcine Respiratory and Reproductive Syndrome (PRRS): A review, with emphasis on pathological, virological and diagnostic aspects. British Veterinary Journal 152 (2): 153-174.

17. Drew, T. W., Meulenberg, J. j. M., Sands, J. J. and Paton, D. J. 1995. Production, characterization and reactivity of monoclonal antibodies to porcine reproductive and respiratory syndrome virus. J. Gen. Virol. 76:1361-1369.

18. Edbauer, C., R. Weinberg, J. Taylor, A. Rey-Senelonge, J. F. Bouquet, P. Desmettre and E. Paoletti, Virology 179, 901-904 (1990).

19. Faaberg, K. S. and Plagemann, P. G. W. 1995. The envelope proteins of lactate dehydrogenase-elevating virus and their membrane topography. Virology 212:512-525.

20. Faaberg, K. S., Even, C., Palmer, G. A. and Plagemann, P. G. W. 1995. Disulfide bonds between two envelope proteins of lactate dehydrogenase-elevating virus are essential for viral infectivity. J. Virol. 69:613-617.

21. Galina, L., Pijoan, C., Sitjar, M., Christianson, W. T., Rossow, K. and Collins, J. E. 1994.

Interaction between Streptococcus suis serotype 2 and porcine reproductive and respiratory syndrome virus in specific pathogen-free piglets. Vet. Record. 134:60-64.

22. Godeny, E. K., Chen, L., Kumar, S. N., Methven, S. L., Koonin, E. V., and Brinton, M. A. 1993. Complete genomic sequence and phylogenetic analysis of the lactate dehydrogenase elevating virus (LDV). Virology 192:585-596.

23. Goebel, S. J., G. P. Johnson, M. E. Perkus, S. W. Davis, J. P. Winslow, E. Paoletti, Virology 179, 247-266, 517-563 (1990).

24. Gonin P, Mardassi H., Gagnon C A, Massie B., Dea S. 1999. A nonstructural and antigenic glycoprotein is encoded by ORF3 of the IAF-Klop strain of porcine reproductive and respiratory syndrome virus. Arch. Virol. 143:1927-1940.

25. Gonin P, Pirzadeh B, Gagnon C A, Dea S. 1999. Seroneutralization of porcine reproductive and respiratory syndrome virus correlates with antibody response to the GP5 major envelope glycoprotein. J Vet Diagn Invest 11:20-26.

26. Gorcyca, D., Schlesinger, K., Chladek, D. and Behan, W. 1995. RespPRRS: a new tool for the prevention and control of PRRS in pigs. In Proceedings of the American Association of Swine Practitioners, pp1-22

27. Guo, P., S. Goebel, S. Davis, M. E. Perkus, B. Languet, P. Desmettre, G. Allen, and E. Paoletti, J. Virol. 63, 4189-4198 (1989).

28. Halbur, P. G., Paul, P. S., Meng, X. and Andrews, J. J. 1992. Comparative pathology of porcine reproductive and respiratory syndrome in SPF pigs. Iowa State University Swine Research Reports, p137. Cooperative Extension Service, Iowa State University, Ames, Iowa, 50011.

29. Halbur, P. G., Paul, P. S. and Janke, B. H. 1993. Viral contributors to the porcine respiratory disease complex. In Proceedings of the 24^{th} Annual meeting of the American Association of swine Practitioners, Kansas City, Mo., USA, pp343-350.

30. Halbur, P. G., Paul, P. S., Frey, M. L.,Landgraf, J., Eernisse, K., Meng, X.-J., Lum, M. A., Andrews, J. J. and Rathje, J. A. 1995. Comparison of the pathology of two U.S. porcine reproductive and respiratory syndrome virus isolates with the Lelystad virus. Vet. Pathol. 32:648-660.

31. Heinen E, Herbst W, Schmeer N. 1998. Isolation of a cytopathogenic virus from a case of porcine reproductive and respiratory syndrome (PRRS) and its characterization as parainfluenza virus type 2. Arch Virol 143:2233-2239

32. Hill, H. 1990. Overview and history of mystery swine disease (swine infertility and respiratory syndrome). In Proceedings of the Mystery Swine Disease Committee Meeting, Denver, Colo., 1990. Livestock Conservation Institute, Madison, Wis., pp 29-30.

33. Kwang J, Zuckermann F, Ross G, Yang S, Osorio F, Liu W, Low S. 1999. Antibody and cellular immune responses of swine following immunisation with plasmid DNA encoding the PRRS virus ORF's 4, 5, 6 and 7. Res Vet Sci 67:199-201.

34. Lager KM, Mengeling WL, Brockmeier SL. 1999. Evaluation of protective immunity in gilts inoculated with the NADC-8 isolate of porcine reproductive and respiratory syndrome virus (PRRSV) and challenge-exposed with an antigenically distinct PRRSV isolate. Am J Vet Res 60:1022-1027.

35. Le Potier, M. F., Blanquefort, P., Morvan, E. and Albina, E. 1995. Results of a control program for PRRS in the French area ‘Pays de Loire’. Proc. of the 2^{nd} Int. Symposium on PRRS, Copenhagen, Denmark, 9-10 August, p34.

36. Mardassi, H., Mounir, S. and Dea, S. 1995. Molecular analysis of the ORFs 3 to 7 of porcine reproductive and respiratory syndrome virus, Quebec reference strain. Arch. Virol. 140:1405-1418.

37. Mardassi, H., Massie, B. and Dea, S. 1996. Intracellular synthesis, processing, and transport of proteins encoded by ORFs 5 to 7 of porcine reproductive and respiratory syndrome. Virology 221:98-112.

38. Meng, X.-J., Paul, P.S., Halbur, P. G. and Lunn, M. A. 1995a. Phylogenetic analysis of the putative M (ORF6) and N(ORF7) genes of porcine reproductive and respiratory syndrome virus (PRRSV): implications for the existence of two genotypes of PRRSV in the USA and Europe. Arch. Virol. 140:745-755.

39. Meng, X.-J., Paul, P. S., Halbur, P. G. and Morozov, I. 1995b. Sequence comparison of open reading frames 2 to 5 of low and high virulence United States isolates of porcine reproductive and respiratory syndrome virus. J. Gen. Virol. 76:3181-3188.

40. Meng, X.-J., Paul, P. S., Morozov, I. And Halbur, P. G. 1996. A nested set of six or seven subgenomic mRNAs is formed in cells infected with different isolates of porcine reproductive and respiratory syndrome virus. J. Gen. Virol. 77; 1265-1270.

41. Mengeling W L, Vorwald A C, Lager K M, Clouser D F, Wesley R D. 1999a. Identification and clinical assessment of suspected vaccine-related field strains of porcine reproductive and respiratory syndrome virus. Am J Vet Res 60:334-340.

42. Mengeling W L, Lager K M, Vorwald A C. 1999b. Safety and efficacy of vaccination of pregnant gilts against porcine reproductive and respiratory syndrome. Am J Vet Res 60:796-801.

43. Mengeling WL, Lager KM, Vorwald AC. 1998. Clinical consequences of exposing pregnant gilts to strains of porcine reproductive and respiratory syndrome (PRRS) virus isolated from field cases of “atypical” PRRS. Am J Vet Res 59:1540-1544.

44. Meulenberg, J. J. M., Hulst, M. M., de Meijer, E. J., Moonen, P. J. L. M., den Besten, A., de Kluyver, E. P., Wensvoort, G. and Moormann, R. J. M. 1993a. Lelystad virus, the causative agent of porcine epidemic abortion and respiratory syndrome (PEARS), is related to LDV and EAV. Virology 192:62-72.

45. Meulenberg, J. J. M., de Meijer, E. J. and Moormann, R. J. M. 1993b. Subgenomic RNAs of Lelystad virus contain a conserved junction sequence. J. Gen. Virol. 74:1697-1701.

46. Meulenberg, J. J. M., den Besten, A. P., De Kluyver, E. P., Moormann, R. J. M., Schaaper, W. M. M. and Wensvoort, G. 1995. Characterization of proteins encoded by ORFs 2 to 7 of Lelystad virus. Virology 206:155-163.

47. Meulenberg, J. J. M. and Petersen-Den Besten, P.-D. 1996. Identification and characterization of a sixth structural protein of Lelystad virus: The glycoprotein GP₂ encoded by ORF 2 is incorporated in virus particles. Virology 225:44-51.

48. Murtaugh, M. P., Elam, M. R. and Kakach, L. T. 1995 Comparison of the structural protein coding sequences of the VR-2332 and Lelystad virus strains of PRRS virus. Arch. Virol. 140:1451-1460.

49. Nakamine M, Kono Y, Abe S, Hoshino C, Shirai J, Ezaki T. 1998. Dual infection with enterotoxigenic Escherichia coli and porcine reproductive and respiratory syndrome virus observed in weaning pigs that died suddenly. J Vet Med Sci 60:555-561.

50. Nelsen CJ, Murtaugh M P, Faaberg K S. 1999. Porcine reproductive and respiratory syndrome virus comparison: divergent evolution on two continents. J Virol 73:270-280.

51. Nelson, E. A., Christopher-Hennings, Drew, T., Wensvoort, G., Collins, G. and Benfield, D.A. 1993. Differentiation of US and European isolates of porcine reproductive nad respiratory syndrome virus by monoclonal antibodies. J. Clin. Micro. 31:3184-3189.

52. Ohlinger, V., Haas, B., Saalmuller, A., Beyer, J., Teuffert, J., Visser, N. and Weiland, F. 1992. In vivo and in vitro studies on the immunobiology of PRRS. Proc. of American Assoc. Swine Practitioners-1^{st} Int. PRRS Symp., 4(4): 24.

53. Ohlinger V F. 1995. The respiratory syndrome: studies on PRRSV-replication and immune response. Int. Symp. PRRS 2:12.

54. Panicali, D. and E. Paoletti, Proc. Natl. Acad. Sci. USA 79, 4927-4931 (1982).

55. Pirzadeh B, Dea S. 1998a. Immune response in pigs vaccinated with plasmid DNA encoding ORF5 of porcine reproductive and respiratory syndrome virus. J Gen Virol 79:989-999.

56. Pirzadeh B, Gagnon C A, Dea S. 1998b. Genomic and antigenic variations of porcine reproductive and respiratory syndrome virus major envelope GP5 glycoprotein. Can J Vet Res 62:170-177

57. Paoletti, E., B. R. Lipinskaks, C. Samsonoff, S. Mercer, and D. Panicali, Proc. Natl. Acad. Sci. U.S.A. 81, 193-197 (1984).

58. Paton, D. J., Brown, I. H., Edwards, S. and Wensvoort, G. 1991. Blue ear disease of pigs.

Vet. Rec. 128:617.

59. Perkus, M. E., K. Limbach, and E. Paoletti, J. Virol. 63, 3829-3836 (1989).

60. Piccini, A., M. E. Perkus, and E. Paoletti, In Methods in Enzymology, Vol. 153, eds. Wu, R., and Grossman, L., (Academic Press) pp. 545-563 (1987).

61. Pirzadeh, B. and Dea, S. 1997. Monoclonal antibodies to the ORF5 product of porcine reproductive and respiratory syndrome virus define linear neutralizing determinants. J. Gen. Virol. 78:1867-1873.

62. Plagemann, P. G. W. 1996. Lactate dehydrogenase-elevating virus and related viruses. In “Virology” (B. N. Fields, D. M. Knipe and P. M. Howley, Eds.) 3^{rd} ed., pp1105-1120. Raven Press, New York.

63. Plana-Duran J, Bastons M, Urniza A, Vayreda M, Vila X, Mane H. 1997. Efficacy of an inactivated vaccine for prevention of reproductive failure induced by porcine reproductive and respiratory syndrome virus. Vet Microbiol 55:361-370.

64. Plana Duran, J., Climent, I., Sarraseca, J., Urniza, A., Cortes, E., Vela, C. and Casal, I. 1997. Baculovirus expression of proteins of porcine reproductive and respiratory syndrome virus strain Olot/91. Involvement of ORF3 and ORF5 proteins in protection. Virus Genes 14:19-29.

65. Rossow K. D. 1998. Porcine reproductive and respiratory syndrome (review article). Vet Pathol. 35:1-20.

66. Sirinarumitr T, Zhang Y, Kluge J P, Halbur P G, Paul P S. 1998. A pneumo-virulent United States isolate of porcine reproductive and respiratory syndrome virus induces apoptosis in bystander cells both in vitro and in vivo. J Gen Virol 79:2989-2995.

67. Snijder E., van Tol H., Pedersen K. W., Raamsman M. J. B., and de Vries A. A. F. 1999. Identification of a novel structural protein of arteriviruses. J. Virol. 73, 6335-6345.

68. Suarez, P., Diaz-Guerra, M., Prieto, C., Esteban, M., Castro, J. M., Nieto, A. and Ortin, J. 1996. Open reading frame 5 of porcine reproductive and respiratory syndrome virus as a cause of virus-induced apoptosis. J. Virol. 70:2876-2882.

69. Sur J H, Doster A R, Osorio F A. 1998. Apoptosis induced in vivo during acute infection by porcine reproductive and respiratory syndrome virus. Vet Pathol 35:506-514.

70. Tartaglia, J., J. Winslow, S. Goebel, G. P. Johnson, J. Taylor, and E. Paoletti, J. Gen. Virol. 71, 1517-1524 (1990).

71. Tartaglia, J., Perkus M E, Taylor J, Norton E K, Audonnet J C, Cox W I, Davis S W, van der Hoeven J, Meignier B, Riviere M, and E. Paoletti, Virology 188, 217-32 (1992).

72. Taylor, J., R. Weinberg, B. Languet, Ph. Desmettre and E. Paoletti, Vaccine 6, 497-503 (1988a).

73. Taylor, J. and E. Paoletti, Vaccine 6, 466-468 (1988b).

74. Taylor, J., R. Weinberg, Y. Kawaoka, R. Webster and E. Paoletti, Vaccine 6, 504-508 (1988c).

75. Taylor, J., C. Edbauer, A. Rey-Senelonge, J. F. Bouquet, E. Norton, S. Goebel, P. Desmettre and E. Paoletti, J. Virol. 64, 1441-1450 (1990).

76. Taylor, J., C. Trimarchi, R. Weinberg, B. Languet, F. Guillemin, P. Desmettre and E. Paoletti, Vaccine 9, 190-193 (1991).

77. Taylor J, Weinberg R, Tartaglia J, Richardson C, Alkhatib G, Briedis D, Appel M, Norton E, Paoletti E., Virology187, 321-328 (1992).

78. Thacker E L, Halbur P G, Ross R F, Thanawongnuwech R, Thacker B J. 1999. Mycoplasma hyopneumoniae potentiation of porcine reproductive and respiratory syndrome virus-induced pneumonia. J Clin Microbiol 37:620-627.

79. Van Nieuwstadt, A. P., Meulenberg, J. J. M., van Essen-Zandbergen, A., Petersen-den Besten, A., Bende, R. J., Moorman, R. J. M. and Wensvoort, G. 1996. Proteins encoded by open reading frames 3 and 4 of the genome of Lelystad virus (Arteriviridae) are structural proteins of the virion. J. Virol. 70:4767-4772.

80. van Woensel P A, Liefkens K, Demaret S. 1998a. Effect on viraemia of an American and a European serotype PRRSV vaccine after challenge with European wild-type strains of the virus. Vet Rec 142:510-512.

81. van Woensel PA, Liefkens K, Demaret S. 1998b. European serotype PRRSV vaccine protects against European serotype challenge whereas an American serotype vaccine does not. Adv Exp Med Biol 440:713-718.

82. Weiland E, Wieczorek-Krohmer M, Kohl D, Conzelmann K K, Weiland F. 1999.

Monoclonal antibodies to the GP5 of porcine reproductive and respiratory syndrome virus are more effective in virus neutralization than monoclonal antibodies to the GP4. Vet Microbiol 66:171-186

83. Wensvoort, G. C., Terpstra, J. M. A., Pol, E. A., ter Laak, M., Bloemraad, E. P., de Kluyver, C., Kragten, C., van Buiten, A., den Besten, F., Wagenaar, J. M., Broekhuysen, P. L. J. M., Moonen, T., Zetstra, E. A., de Boer, H. J., Tibben M. F., de Jong, P., van't Veld, G. J. R., Groenland, .A., van Gennep, M. T., Voets, J. H. M., Verheyden, J. H. M. and Braamskamp, J. 1991. Mystery swine disease in The Netherlands: the isolation of Lelystad virus. Vet Q. 13:121-130.

84. Wensvoort, G., de Kluyver, E. P., Luijtze, E. A., den Besten, A., Harris, L., Collins, J. E., Christianson, J. E. and Chladek, D. 1992. Antigenic comparison of Lelystad virus and swine infertility and respiratory syndrome (SIRS) virus. J. Vet. Diagn. Invest. 4:134-138.

85. Yeager, M. J., Prieve, T., Collins, J., Christopher-Hennings, J., Nelson, E. and Benfield, D. 1993. Evidence for the transmission of porcine reproductive and respiratory syndrome (PRRS) virus in boar semen. Swine Health Prod. 1(5): 7-9.

86. Yoon, K.-J., Zimmermann, J. J., Swenson, S. L., Wills, R. W., Hill, H. T. and Platt, K. B. 1994. Assessment of the biological significance of antibody dependent enhancement (ADE) of porcine epidemic abortion and respiratory syndrome (PEARS) virus infection in passively immunized pigs. Proc. 13ₜₕ Int. Pig Vet. Soc. Congress, p69.

87. Yoon, K.-J., Zimmerman, J. J., Swenson, S. L., McGinley, M. J., Eernisse, K. A., Brevik, A., Rhinehart, L. L., Frey, M. L., Hill, H. T. and Platt, K. B. 1995. Characterization of the humoral immune response to porcine reproductive and respiratory syndrome (PRRS) virus infection. J. Vet. Diagn. Invest. 7:305-312.

88. Zimmermann, J. J., Yoon, K.-J., Wills, R. W. and Swenson, S. L. 1997. General Overview of PRRSV: A perspective from the United States. Veterinary Microbiology 55:187-196.

We claim:

1. A recombinant avipox virus comprising heterologous DNA wherein the heterologous DNA comprises DNA complementary to genomic RNA from porcine reproductive and respiratory syndrome virus (PRRSV).

2. The recombinant avipox virus of claim 1 which is a fowlpox virus recombinant virus.

3. The recombinant avipox virus of claim 1 which is a canarypox virus recombinant virus.

4. The recombinant avipox virus of claim 1 which is ALVAC recombinant virus.

5. The recombinant avipox virus according to claim 1, wherein the heterologous DNA comprises DNA that codes for and is expressed as one or more proteins selected from the group consisting of: one or more PRRSV glycoproteins, one or more PRRSV membrane proteins, one or more PRRSV capsid proteins, and combinations thereof.

6. The recombinant avipox virus according to claim 2, wherein the heterologous DNA comprises DNA that codes for and is expressed as one or more proteins selected from the group consisting of: one or more PRRSV glycoproteins, one or more PRRSV membrane proteins, one or more PRRSV capsid proteins, and combinations thereof.

7. The recombinant avipox virus according to claim 3, wherein the heterologous DNA comprises DNA that codes for and is expressed as one or more proteins selected from the group consisting of: one or more PRRSV glycoproteins, one or more PRRSV membrane proteins, one or more PRRSV capsid proteins, and combinations thereof.

8. The recombinant avipox virus according to claim 4, wherein the heterologous DNA comprises DNA that codes for and is expressed as one or more proteins selected from the group consisting of: one or more PRRSV glycoproteins, one or more PRRSV membrane proteins, one or more PRRSV capsid proteins, and combinations thereof.

9. The recombinant avipox virus of claim 5, wherein the heterologous DNA comprises DNA complementary to PRRSV open reading frame 5 (ORF5); or DNA complementary to PRRSV open reading frames 5 (ORF5) and 3 (ORF3); or DNA complementary to PRRSV open reading frames 5 (ORF5) and 6 (ORF6); or DNA complementary to PRRSV open reading frames 5 (ORF5) and 3 (ORF3) and 6 (ORF6).

10. The recombinant avipox virus of claim 6, wherein the heterologous DNA comprises DNA complementary to PRRSV open reading frame 5 (ORF5); or DNA complementary to PRRSV open reading frames 5 (ORF5) and 3 (ORF3); or DNA complementary to PRRSV open reading frames 5 (ORF5) and 6 (ORF6); or DNA complementary to PRRSV open reading frames 5 (ORF5) and 3 (ORF3) and 6 (ORF6).

11. The recombinant avipox virus of claim 7, wherein the heterologous DNA comprises DNA complementary to PRRSV open reading frame 5 (ORF5); or DNA complementary to PRRSV open reading frames 5 (ORF5) and 3 (ORF3); or DNA complementary to PRRSV open reading frames 5 (ORF5) and 6 (ORF6); or DNA complementary to PRRSV open reading frames 5 (ORF5) and 3 (ORF3) and 6 (ORF6).

12. The recombinant avipox virus of claim 8, wherein the heterologous DNA comprises DNA complementary to PRRSV open reading frame 5 (ORF5); or DNA complementary to PRRSV open reading frames 5 (ORF5) and 3 (ORF3); or DNA complementary to PRRSV open reading frames 5 (ORF5) and 6 (ORF6); or DNA complementary to PRRSV open reading frames 5 (ORF5) and 3 (ORF3) and 6 (ORF6).

13. The recombinant avipox virus of claim 9, wherein the heterologous DNA comprises DNA complementary to ORF5 and ORF3 in a head-to-head orientation.

14. The recombinant avipox virus of claim 10, wherein the heterologous DNA comprises DNA complementary to PRRSV ORF5 and ORF3 in a head-to-head orientation.

15. The recombinant avipox virus of claim 11, wherein the heterologous DNA comprises DNA complementary to PRRSV ORF5 and ORF3 in a head-to-head orientation.

16. The recombinant avipox virus of claim 12, wherein the heterologous DNA comprises DNA complementary to PRRSV ORF5 and ORF3 in a head-to-head orientation.

17. The recombinant avipox virus of claim 9, wherein the heterologous DNA comprises DNA complementary to PRRSV ORF5 and ORF3 and ORF6, with the DNA complementary to PRRSV ORF5 and ORF6 or the DNA complementary to PRRSV ORF5 and ORF3 in a head-to-head orientation.

18. The recombinant avipox virus of claim 10, wherein the heterologous DNA comprises DNA complementary to PRRSV ORF5 and ORF3 and ORF6, with the DNA complementary to PRRSV ORF5 and ORF6 or the DNA complementary to PRRSV ORF5 and ORF3 in a head-to-head orientation.

19. The recombinant avipox virus of claim 11, wherein the heterologous DNA comprises DNA complementary to PRRSV ORF5 and ORF3 and ORF6, with the DNA complementary to PRRSV ORF5 and ORF6 or the DNA complementary to PRRSV ORF5 and ORF3 in a head-to-head orientation.

20. The recombinant avipox virus of claim 12, wherein the heterologous DNA comprises DNA complementary to PRRSV ORF5 and ORF3 and ORF6, with the DNA complementary to PRRSV ORF5 and ORF6 or the DNA complementary to PRRSV ORF5 and ORF3 in a head-to-head orientation.

21. The recombinant avipox virus of claim 4 which is vCP1618, vCP1619, vCP1626, or vCP1643.

22. An immunological or vaccine composition
(a) for inducing an immunological response against PRRSV in a host inoculated with the composition; or
(b) for inducing an immunological response against PRRSV and at least one other porcine pathogen in a host inoculated with the composition;
wherein: composition (a) comprises at least one recombinant virus and a carrier; composition (b) comprises (i) at least one recombinant virus, (ii) at least one antigen or epitope of at least one porcine pathogen other than PRRSV, or a vector expressing at least one antigen or epitope or of at least one porcine pathogen other than PRRSV, and (iii) a carrier; the at least one recombinant virus in compositions (a) and (b) comprises at least one recombinant virus according to any one of claims 1-21; and each of compositions (a) and (b) optionally further comprises an adjuvant.

23. The composition of claim 22, wherein the adjuvant is present and is a polymer of acrylic or methacrylic acid or a copolymer of maleic anhydride and alkenyl derivative; or, a carbomer; or has the formula. 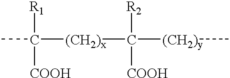
in which:
R₁ and R₂, are identical or different, and represent H or CH₃
x=0 or 1, and
y=1 or 2,with x+y=2.

24. The composition of claim 23 wherein x=0 and y=2; or x=y=1.

25. A method for eliciting an immunological response against PRRSV comprising administering to a host a recombinant virus as claimed in any one of claims 1-21, or a composition comprising the recombinant virus.
